(19) United States
(12) Patent Application Publication
(10) Pub. No.: US 2026/0070948 A1
(43) Pub. Date: Mar. 12, 2026

(54) CYCLIC CELL-PENETRATING PEPTIDES WITH CARGO

(71) Applicant: Ohio State Innovation Foundation, Columbus, OH (US)

(72) Inventors: Dehua PEI, Columbus, OH (US); Marina BUYANOVA, Columbus, OH (US)

(73) Assignee: Ohio State Innovation Foundation, Columbus, OH (US)

(21) Appl. No.: 19/329,440

(22) Filed: Sep. 15, 2025

Related U.S. Application Data
(63) Continuation of application No. 18/278,259.
(60) Provisional application No. 63/152,048, filed on Feb. 22, 2021.

(51) Int. Cl. C07K 7/64; A61K 47/64

(52) U.S. Cl. CPC C07K 7/64; A61K 47/64

(57) ABSTRACT

Provided herein are novel cyclic cell penetrating peptides comprising at least two arginines and at least three hydrophobic amino acids. The disclosure also provides methods of using the cyclic cell penetrating peptides to transport cargo into cells and to treat diseases.

### CROSS-REFERENCE TO RELATED APPLICATIONS

This application is a continuation of U.S. application Ser. No. 18/278,259, filed Aug. 22, 2023, which is a U.S. National Stage application filed under 35 U.S.C. § 371 of PCT/US2022/017211, filed Feb. 22, 2022, which claims the benefit of priority to U.S. Provisional Application No. 63/152,048, filed on Feb. 22, 2021. The entirety of each of these applications is hereby incorporated by reference for all purposes.

### STATEMENT OF GOVERNMENT SUPPORT

This invention was made with government support under GM122459 awarded by the National Institutes of Health. The government has certain rights in the invention.

### INCORPORATION BY REFERENCE

The Sequence Listing XML file named “25063-208US1_ST26” created on Sep. 15, 2025, and having a size of 6,606 bytes, is hereby incorporated by reference.

### BACKGROUND

Traversing the plasma membrane presents a major challenge in drug discovery, especially for biologics such as peptides, proteins and nucleic acids. One potential strategy to subvert the membrane barrier and deliver the biologics into cells is to attach them to cell-penetrating peptides (CPPs, also referred to as endosomal escape vehicles or EEVs). Despite three decades of investigation, the fundamental basis for CPP activity remains elusive. CPPs that enter cells via endocytosis must exit from endocytic vesicles in order to reach the cytosol. Unfortunately, the endosomal membrane has proven to be a significant barrier towards cytoplasmic delivery by these CPPs. What are thus needed are new cell penetrating peptides and compositions comprising such peptides that can be used to deliver agents to various cell types. The compositions and methods disclosed herein address these and other needs.

### SUMMARY

In some embodiments, provided herein is a cyclic peptide, comprising from 6 to 20 amino acids, wherein at least three of the amino acids are arginine and at least three of the amino acids have a hydrophobic side chain selected from an aryl or heteroaryl, wherein the aryl and heteroaryl are optionally substituted.

In some embodiments, provided herein is a cyclic peptide, comprising from 6 to 20 amino acids, wherein at least three of the amino acids are arginine and at least three of the amino acids have a hydrophobic side chain selected from an aryl or heteroaryl, wherein the aryl and heteroaryl are optionally substituted, wherein at least one hydrophobic amino acid has an aryl side chain and at least one hydrophobic amino acid has a heteroaryl side chain.

In some embodiments, provided herein is a cyclic peptide, comprising from 6 to 20 amino acids, wherein at least three of the amino acids are arginine and at least three of the amino acids have a hydrophobic side chain selected from an aryl or heteroaryl, wherein the aryl and heteroaryl are optionally substituted, wherein two hydrophobic amino acids have an aryl side chain and one hydrophobic amino acid has a heteroaryl side chain.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, wherein the hydrophobic amino acid is selected from the group consisting of L-3-benzothienylalanine, L-4-fluorophenylalanine, D-4-fluorophenylalanine, L-1-naphthylalanine, L-2-naphthylalanine, L-2-pyridylalanine, D-2-pyridylalanine, L-4-pyridylalanine, D-4-pyridylalanine, L-phenylalanine, D-phenylalanine, L-tyrosine, D-tyrosine, and combinations thereof.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, wherein the at least three hydrophobic amino acids are L-phenylalanine, D-phenylalanine, and L-3-benzothienylalanine.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, wherein the at least three hydrophobic amino acids are L-phenylalanine, D-4-pyridylalanine, and L-2-napthylalanine.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, wherein at least one hydrophobic amino acid is L-3-benzothienylalanine.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, wherein at least one hydrophobic amino acid is D-4-pyridylalanine.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least one D-amino acid.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least two D-amino acids.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least three D-amino acids.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least two consecutive amino acids having the same chirality.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least two consecutive amino acids having the same chirality, wherein the two consecutive amino acids having the same chirality are arginine and an amino acid having a hydrophobic side chain.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least two consecutive amino acids having alternating chirality.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least three consecutive amino acids having alternating chirality.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least four consecutive amino acids having alternating chirality.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least five consecutive amino acids having alternating chirality.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least two consecutive amino acids having alternating chirality, wherein the at least two consecutive amino acids having alternating chirality are hydrophobic amino acids.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least three consecutive amino acids having alternating chirality, wherein the at least three consecutive amino acids having alternating chirality are hydrophobic amino acids.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least three arginines.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least four arginines.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least two consecutive arginines.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least three consecutive arginines.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least four consecutive arginines.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least two consecutive arginines with alternating chirality.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least three consecutive arginines with alternating chirality.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least four consecutive arginines with alternating chirality.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising a glutamine.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising at least one amino acid selected from the group consisting of cysteine, glutamine, 2,3-diaminopropionic acid, ornithine, lysine, serine, aspartic acid, glutamic acid, asparagine, and tryptophan.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, having a structure of:

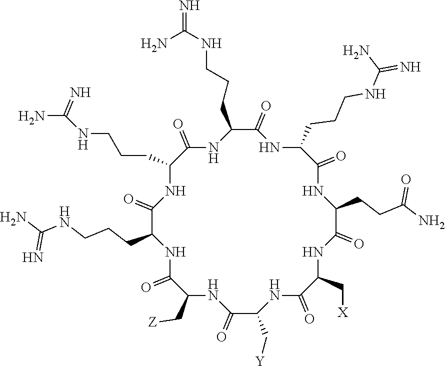

wherein each of X, Y, and Z independently comprise aryl or heteroaryl, wherein the aryl and heteroaryl are optionally substituted. In some embodiments, X, Y, and Z are independently selected from C₆₋₁₄ aryl or C₅-C₁₅ heteroaryl having from 1 to 5 heteroatoms selected from N, O, or S. In some embodiments, two of X, Y, and Z are independently C₆-14 aryl and one of X, Y, and Z are C₅-C₁₅ heteroaryl having from 1 to 5 heteroatoms selected from N, O, or S. In some embodiments, X, Y, and Z are independently phenyl, naphthyl, pyridyl, or benzothienyl. In some embodiments, X, Y, and Z are independently selected from the group consisting of phenyl, pyridyl, and naphthyl. In some embodiments, X is phenyl, Y is phenyl, and Z is benzothienyl. In some embodiments, X is phenyl, Y is pyridyl, and Z is naphthyl. In some embodiments, X, Y, and Z comprise a side chain from an amino acid independently selected from the group consisting of 3-benzothienylalanine, 4-fluorophenylalanine, 1-naphthylalanine, 2-naphthylalanine, 2-pyridylalanine, 4-pyridylalanine, phenylalanine, tyrosine, and combinations thereof.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, having a structure of:

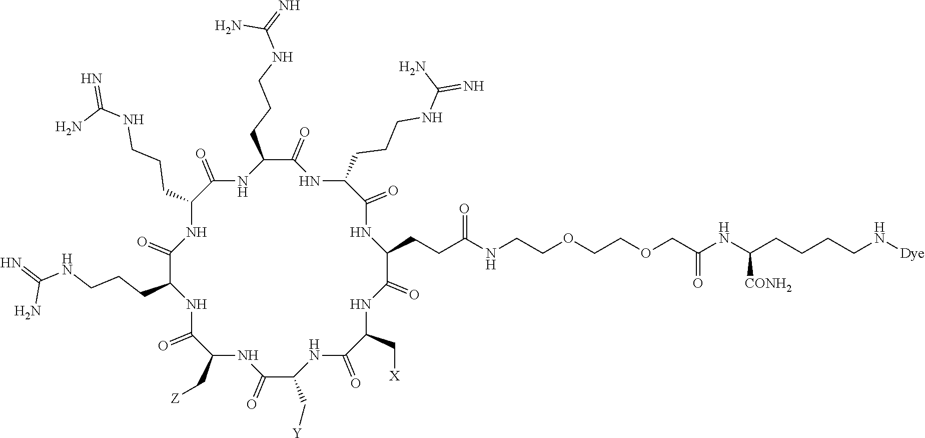

wherein each of X, Y, and Z independently comprise aryl or heteroaryl, wherein the aryl and heteroaryl are optionally substituted. In some embodiments, X, Y, and Z are independently selected from C₆₋₁₄ aryl or C₅-C₁₅ heteroaryl having from 1 to 5 heteroatoms selected from N, O, or S. In some embodiments, two of X, Y, and Z are independently C₆-14 aryl and one of X, Y, and Z are C₅-C₁₅ heteroaryl having from 1 to 5 heteroatoms selected from N, O, or S. In some embodiments, X, Y, and Z are independently phenyl, naphthyl, pyridyl, or benzothienyl. In some embodiments, X, Y, and Z are independently selected from the group consisting of phenyl, pyridyl, and naphthyl. In some embodiments, X is phenyl, Y is phenyl, and Z is benzothienyl. In some embodiments, X is phenyl, Y is pyridyl, and Z is naphthyl. In some embodiments, X, Y, and Z comprise a side chain from an amino acid independently selected from the group consisting of 3-benzothienylalanine, 4-fluorophenylalanine, 1-naphthylalanine, 2-naphthylalanine, 2-pyridylalanine, 4-pyridylalanine, phenylalanine, tyrosine, and combinations thereof.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, having a relative cytosolic delivery efficiency which is improved by about 110% to about 400% compared to cyclo(FfΦRrRrQ).

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, having a relative cytosolic delivery efficiency which is improved by about 375% compared to cyclo(FfΦRrRrQ).

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, having a relative cytosolic delivery efficiency which is improved by about 115% compared to cyclo(FfΦRrRrQ).

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, having an endosomal escape efficiency, γ, which is improved by about 108% to about 400% compared to cyclo(FfΦRrRrQ).

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, having an endosomal escape efficiency, γ, which is improved by about 108% compared to cyclo(FfΦRrRrQ).

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, having an endosomal escape efficiency, γ, which is improved by about 383% compared to cyclo(FfΦRrRrQ).

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, having an amino acid sequence selected from the group consisting of:

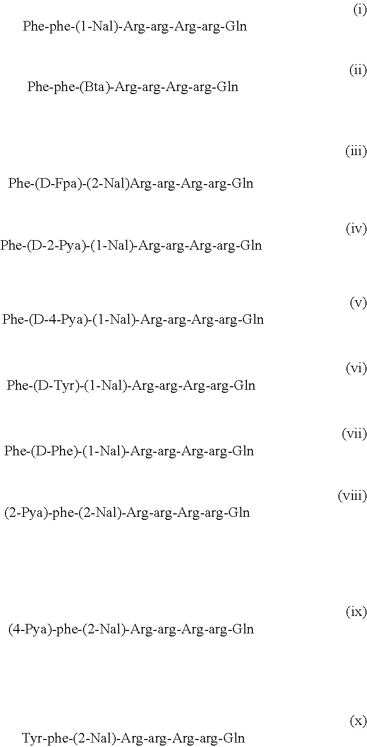

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, having an amino acid sequence of Phe-phe-(Bta)-Arg-arg-Arg-arg-Gln.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, having an amino acid sequence of Phe-(D-4-Pya)-(2-Nal)-Arg-arg-Arg-arg-Gln.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising a cargo.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising a cargo selected from the group consisting of a detectable moiety, a targeting moiety, a therapeutic moiety, or any combination thereof.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising a cargo which is coupled to a side chain of an amino acid of the cyclic peptide.

In some embodiments, provided herein is a cyclic peptide, comprising from 6 to 10 amino acids, wherein at least three of the amino acids are arginine and at least three of the amino acids have a hydrophobic side chain selected from an aryl or heteroaryl, wherein the aryl and heteroaryl are optionally substituted, comprising a cargo, which is coupled to a glutamine side chain of the CPP.

In some embodiments, provided herein is a method of treating a disease or pathology in a subject in need comprising administering to the subject a cyclic peptide comprising from 6 to 10 amino acids, wherein at least three of the amino acids are arginine and at least three of the amino acids have a hydrophobic side chain selected from an aryl or heteroaryl, wherein the aryl and heteroaryl are optionally substituted. In some embodiments, the cyclic peptide comprises a cargo, such as a therapeutic moiety.

In some embodiments, provided herein is a method of treating cancer comprising from 6 to 10 amino acids, wherein at least three of the amino acids are arginine and at least three of the amino acids have a hydrophobic side chain selected from an aryl or heteroaryl, wherein the aryl and heteroaryl are optionally substituted. In some embodiments, the cyclic peptide comprises a cargo, such as a therapeutic moiety.

### BRIEF DESCRIPTION OF THE FIGURES

FIG. 1A shows the cytosolic entry efficiencies of NF-labelled CPP12 and its analogs CPP12-1 to CPP12-10 in Hela cells in the presence of 10% FBS as determined by flow cytometry. MFI values reported are presented after subtraction of background fluorescence and represent the mean±SD of at least three independent experiments and relative to that of CPP12 (100%).

FIG. 1B shows the cytosolic entry efficiencies of NF-labelled CPP12 and its analogs CPP12-1 to CPP12-10 in Hela cells in the presence of 1% FBS as determined by flow cytometry. MFI values reported are presented after subtraction of background fluorescence and represent the mean±SD of at least three independent experiments and relative to that of CPP12 (100%).

FIG. 2A shows the total cellular uptake of TMR-labelled cyclic peptides CPP12 and CPP12-2 in Hela cells in the presence of 10% FBS as determined by flow cytometry. MFI values reported are presented after subtraction of background fluorescence and represent the mean±SD of at least three independent experiments and relative to that of CPP12 (100%).

FIG. 2B shows the total cellular uptake of TMR-labelled cyclic peptides CPP12 and CPP12-2 in Hela cells in the presence of 1% FBS as determined by flow cytometry. MFI values reported are presented after subtraction of background fluorescence and represent the mean±SD of at least three independent experiments and relative to that of CPP12 (100%).

FIG. 3A shows the concentration dependence of the total cellular uptake of TMR-labelled CPP12-2 by HeLa cells in the presence of 1% FBS as determined by flow cytometry. MFI values reported are presented after subtraction of background fluorescence, represent the mean±SD of at least three independent experiments, and are relative to that of CPP12 (100%).

FIG. 3B shows the cytosolic entry efficiency of NF-labelled CPP12-2 by HeLa cells in the presence of 1% FBS as determined by flow cytometry. MFI values reported are presented after subtraction of background fluorescence, represent the mean±SD of at least three independent experiments, and are relative to that of CPP12 (100%).

FIG. 4A shows confocal microscopic images of Hela cells after treatment with 5 μM TMR-labeled CPP12-2 for 2 h in the presence of 1% FBS. I, TMR fluorescence; II, DIC; III, Merge of I and II. Scale bars, 20 μm.

FIG. 4B shows confocal microscopic images of HeLa cells after treatment with 5 μM TMR-labeled CPP12 for 2 h in the presence of 1% FBS. I, TMR fluorescence; II, DIC; III, Merge of I and II. Scale bars, 20 μm.

FIG. 5 shows confocal microscopy images of HeLa cells after treatment with 1.0 or 0.2 mM TMR-labelled CPP12 or CPP12-2 for 2 h in the presence of 1% FBS. Scale bars, 20 μm.

FIG. 6A shows confocal microscopic images of Hela cells after treatment with 5 μM FITC-labeled CPP12-2 for 2 h in the presence of 1% FBS. I, TMR fluorescence; II, DIC; III, Merge of I and II. Scale bars, 20 μm.

FIG. 6B shows confocal microscopic images of HeLa cells after treatment with 5 μM FITC-labeled CPP12 for 2 h in the presence of 1% FBS. I, TMR fluorescence; II, DIC; III, Merge of I and II. Scale bars, 20 μm.

FIG. 6C shows quantitation of the mean fluorescence intensity (MFI) values of HeLa cells after treatment with 5 μM FITC-labeled CPP12-2 or CPP12 for 2 h in the presence of 1% FBS.

FIG. 7A shows binding of FITC-labeled CPP12 and CPP12-2 (c) to proteins in FBS as monitored by FP.

FIG. 7B shows binding of TMR-labeled CPP12 and CPP12-2 (c) to proteins in FBS as monitored by FP.

FIG. 7C shows binding of NF-labeled CPP12 and CPP12-2 to proteins in FBS as monitored by FP.

FIG. 8A shows the structures of CPP12-P1 and CPP12-2-P1.

FIG. 8B shows binding of P1, CPP12-P1 and CPP12-2-P1 to Keap1 as monitored by fluorescence polarization (FP). Keap1 (40 nM), fluorescein-labeled peptide 2 (20 nM), and increasing concentrations of P1, CPP12-P1 and CPP12-2-P1 were incubated for 1 h and FP values were measured and plotted as a function of peptide concentration. Data shown represent the mean±SD of three independent experiments.

FIG. 8C shows induction of luciferase expression in HepG2-ARE (Luc) cells by P1, CPP12-P1, and CPP12-2-P1. Data shown represent the mean±SD of n=5 independent experiments.

FIG. 9 shows the effect of CPP12 and CPP12-2 on the viability of Hela cells as monitored by the MTT assay. Cells were incubated with the peptides for 72 h in the presence of 10% FBS.

### DETAILED DESCRIPTION

### Definitions

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It will also be understood that when a range is provided, said range encompasses each and every value and subrange within the range.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

The terms “peptide,” “protein,” and “polypeptide” are used interchangeably to refer to a natural or synthetic molecule comprising two or more amino acids linked by the carboxyl group of one amino acid to the alpha amino group of another.

As used herein, the term “adjacent” refers to two contiguous amino acids, which are connected by a covalent bond. For example, in the context of a representative cyclic peptide such as

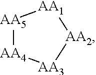

AA₁/AA₂, AA₂/AA₃, AA₃/AA₄, and AA₅/AA₁ exemplify pairs of adjacent amino acids. The term “adjacent” can also be applied to amino acids in a linear sequence, i.e, an acyclic peptide. The term “adjacent” is used interchangeably with consecutive.

A residue of a chemical species, as used herein, refers to a derivative of a moiety that is present in a particular product. To form the product, at least one atom of the moiety is replaced by a bond to a second moiety, such that the product contains a derivative of a moiety. For example, in some embodiments, an aromatic residue in a product may refer to one or more —(C₆H₅)ₙ units present in a cyclic peptide described herein. Similarly, an amino acid residue in a product may refer to cyclic peptide described herein having an amino acid incorporated therein through formation of one or more peptide bonds, and such residues may be referred to interchangeably herein as an amino acid or an amino acid residue.

As used herein, the term “chirality” refers to the “D” and “L” isomers of amino acids or amino acid residues.

As used herein, the term “non-aromatic hydrophobic” refers to a moiety that is not soluble in water and which does not comprise an aromatic ring. Generally, neutral moieties and/or non-polar moieties, or moieties that are predominately neutral and/or non-polar are hydrophobic. Hydrophobic can be measured by one of the methods disclosed herein below. Non-aromatic hydrophobic residues include saturated and unsaturated carbocyclyl and heterocyclyl groups which are not aromatic, as well as alkyl, alkenyl, and alkynyl. In some embodiments, the term “non-aromatic hydrophobic” can include groups in which a hydrophobic residue to attached to rest of the molecule through a bonding group which otherwise could be considered to be polar, such as acyl and alkylcarboxamidyl groups as defined below.

As used herein “aromatic” refers to an unsaturated cyclic molecule having 4n+2π electrons, wherein n is any integer. The term “non-aromatic” refers to any unsaturated cyclic molecule which does not fall within the definition of aromatic.

The term “acyl” refers to groups —C(O)R, where R is hydrogen, alkyl, alkenyl, alkynyl, carbocyclyl, or heterocyclyl, as defined herein. Unless stated otherwise specifically in the specification, acyl can be optionally substituted.

“Alkyl” or “alkyl group” refers to a fully saturated, straight or branched hydrocarbon chain radical having from one to forty carbon atoms, and which is attached to the rest of the molecule by a single bond. Alkyls comprising any number of carbon atoms from 1 to 20 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅alkyl. A C₁-C₅alkyl includes C₅ alkyls, C₄ alkyls, C₃ alkyls, C₂ alkyls and C₁ alkyl (i.e., methyl). A C₁-C₆ alkyl includes all moieties described above for C₁-C₅alkyls but also includes C₆ alkyls. A C₁-C₁₀ alkyl includes all moieties described above for C₁-C₅alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, 1-butyl, n-pentyl, 1-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted.

“Alkenyl” or “alkenyl group” refers to a straight or branched hydrocarbon chain radical having from two to forty carbon atoms, and having one or more carbon-carbon double bonds. Each alkenyl group is attached to the rest of the molecule by a single bond. Alkenyl group comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” refers to the group —OR, where R is alkyl, alkenyl, alkynyl, cycloalkyl, or heterocyclyl as defined herein. Unless stated otherwise specifically in the specification, alkoxy can be optionally substituted.

“Alkylcarbamoyl” refers to the group —O—C(O)—NRₐR_{b}, where Rₐ and R_{b} are the same or different and independently an alkyl, alkenyl, alkynyl, aryl, heteroaryl, cycloalkyl, or heterocyclyl group, as defined herein, or RₐR_{b} can be taken together to form a heterocyclyl group, as defined herein. Unless stated otherwise specifically in the specification, alkylcarbamoyl can be optionally substituted.

“Alkylcarboxamidyl” refers to the group —C(O)—NRₐR_{b}, where Rₐ and R_{b} are the same or different and independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, alkylcarboxamidyl can be optionally substituted.

“Alkoxycarbonyl” refers to the group —C(O)OR, where R is alkyl, alkenyl, alkynyl, aryl, heteroaryl, cycloalkyl, cycloalkenyl, cycloalkynyl, or heterocyclyl group, as defined herein. Unless stated otherwise specifically in the specification, alkoxycarbonyl can be optionally substituted.

“Alkylthio” refers to the —SR or —S(O)ₙ₌₁₋₂—R, where R is alkyl, alkenyl, alkynyl, cycloalkyl, cycloalkenyl, cycloalkynyl, or hetereocyclyl, as defined herein. Unless stated otherwise specifically in the specification, alkylthio can be optionally substituted.

“Arylthio” refers to the —SR or —S(O)ₙ₌₁₋₂—R, where R is aryl or hetereoaryl, as defined herein. Unless stated otherwise specifically in the specification, arylthio can be optionally substituted.

“Alkynyl” or “alkynyl group” refers to a straight or branched hydrocarbon chain radical having from two to forty carbon atoms, and having one or more carbon-carbon triple bonds. Each alkynyl group is attached to the rest of the molecule by a single bond. Alkynyl group comprising any number of carbon atoms from 2 to 40 are included. An alkynyl group comprising up to 40 carbon atoms is a C₂-C₄₀ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain, having from two to forty carbon atoms, and having one or more carbon-carbon triple bonds. Non-limiting examples of C₂-C₄₀ alkynylene include ethynylene, propargylene and the like. Unless stated otherwise specifically in the specification, an alkynylene chain can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Unless stated otherwise specifically in the specification, the carbocyclyl can be a monocyclic, bicyclic, tricyclic or tetracyclic ring system, which can include fused or bridged ring systems Carbocyclic rings include cycloalkyl, cycloalkenyl, and cycloalkynyl as defined herein. In some embodiments, the carbocyclyl is monovalent and is attached to the rest of molecule through a single bond. In some embodiments, the carbocyclyl is divalent and is independently attached to two moieties through single bonds. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Heterocyclyl,” “heterocyclic ring” or “heterocycle” refers to a stable 3- to 20-membered non-aromatic ring radical, which consists of two to fourteen carbon atoms and from one to eight heteroatoms selected from the group consisting of nitrogen, oxygen and sulfur. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocyclyl is monovalent and is attached to the rest of molecule through a single bond. In some embodiments, the heterocyclyl is divalent and is independently attached to two moieties through single bonds. Unless stated otherwise specifically in the specification, a heterocyclyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aryloxy” refers to groups —OAr, where Ar is an aryl or heteroaryl group as defined herein. Unless otherwise stated specifically in the specification, the aryloxy group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“Aralkyl” refers to a radical of the formula —R_{b}—R_{c} where R_{b} is an alkylene, alkenylene or alkynylene group as defined above and R_{c} is one or more aryl radicals as defined above, for example, benzyl, diphenylmethyl and the like. Unless stated otherwise specifically in the specification, an aralkyl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents.

### Cell Penetrating Peptides

Disclosed herein are cyclic peptides having activity as cell penetrating peptides (cCPPs). In some embodiments, the cCPPs include any combination of at least three arginines and either at least three amino acids have a hydrophobic side chain selected from an aryl or heteroaryl, wherein the aryl and heteroaryl are optionally substituted, with a total number of amino acids in the cCPP in the range of from 6 to about 20 amino acids. In some embodiments, the cCPPs disclosed herein comprise about 4 to about to about 13 amino acids, e.g., about 5, about 6, about 7, about 8, about 9, about 10, or about 11 amino acids, or about 12 amino acids, inclusive of all ranges and subranges therebetween. In particular embodiments, the cCPPs disclosed herein comprise from about 5 to about 12 amino acids, from about 6 to about 10 amino acids, or from about 6 to about 8 amino acids.

Each amino acid can be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Thus, as used herein, the term “amino acid” refers to natural and non-natural amino acids, and analogs and derivatives thereof. Examples of suitable amino acids include, but are not limited to, alanine, allosoleucine, arginine, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative, or combinations thereof. Analogs of amino acids encompass that have a structural similar but not identical to an amino acid, e.g., due to a modification to the side chain or backbone on said amino acid. Such modifications may increase the hydrophobicity of the side chain, including elongation of the side chain by one or more hydrocarbons, or increasing the the solvent accessible surface area (SASA as described herein) of an amino acid having an aromatic ring on its side chain, e.g., by conjugating a second aromatic ring or increasing the size of the aromatic ring. Derivatives of amino acids encompass natural and non-natural amino acids that have been modified (e.g., by susbstitution) to include a hydrophobic group as described herein. For example, a derivative of lysine includes lysine whose side chain has been substituted with alkylcarboxamidyl. These, and others, are listed in the Table 1 along with their abbreviations used herein.

TABLE 1
Amino Acid Abbreviations

| | Abbreviations* | Abbreviations* |
|---|---|---|
| Amino Acid | L-amino acid | D-amino acid |
| Alanine | Ala (A) | ala (a) |
| Allosoleucine | AIle | Aile |
| Arginine | Arg (R) | arg (r) |
| Asparagine | Asn (N) | asn (n) |
| Aspartic acid | Asp (D) | asp (d) |
| Cysteine | Cys (C) | cys (c) |
| Cyclohexylalanine | Cha | Cha |
| 2,3-diaminopropionic acid | Dap | Dap |
| 4-fluorophenylalanine | Fpa (Σ) | Pfa |
| Glutamic acid | Glu (E) | glu (e) |
| Glutamine | Gln (Q) | gln (q) |
| Glycine | Gly (G) | gly (g) |
| Gistidine | His (H) | his (h) |
| Homoproline (aka pipecolic acid) | Pip (Θ) | pip (θ) |
| Isoleucine | Ile (I) | ile (i) |
| Leucine | Leu (L) | leu (l) |
| Lysine | Lys (K) | lys (k) |
| Methionine | Met (M) | met (m) |
| Napthylalanine | Nal (Φ) | nal (φ) |
| Norleucine | Nle (Ω) | nle |
| Phenylalanine | Phe (F) | phe (f) |
| Phenylglycine | Phg (Ψ) | phg |
| 4-(phosphonodifluoromethyl)phenylalanine | F₂Pmp (Λ) | f₂pmp |
| Proline | Pro (P) | pro (p) |
| Sarcosine | Sar (Ξ) | sar |
| Selenocysteine | Sec (U) | sec (u) |
| Serine | Ser (S) | ser (s) |
| Threonine | Thr (T) | thr (y) |
| Tyrosine | Tyr (Y) | tyr (y) |
| Tryptophan | Trp (W) | trp (w) |
| Valine | Val (V) | val (v) |
| 3-(3-benzothienyl)-alanine | Bta | Bta |
| 4-fluorophenylalanine | Fpa or L-Fpa | fpa or D-Fpa |
| 1-naphthylalanine | L-1-Nal or 1-Nal | D-1-Nal or 1-nal |
| 2-naphthylalanine | L-2-Nal or 2-Nal | D-2-Nal or 2-nal |
| 2-pyridylalanine | L-2-Pya or 2-Pya | D-2-Pya or 2-pya |
| 4-pyridylalanine | L-4-Pya or 4-Pya | D-4-Pya or 4-pya |

*single letter abbreviations: when shown in capital letters herein it indicates the L-amino acid form, when shown in lower case herein it indicates the D-amino acid form.

cCPPs with at Least Three Amino Acids Having a Hydrophobic Side Chain

In various embodiments, the cCPPs disclosed herein have a structure according to one of Formula I-A to I-F:

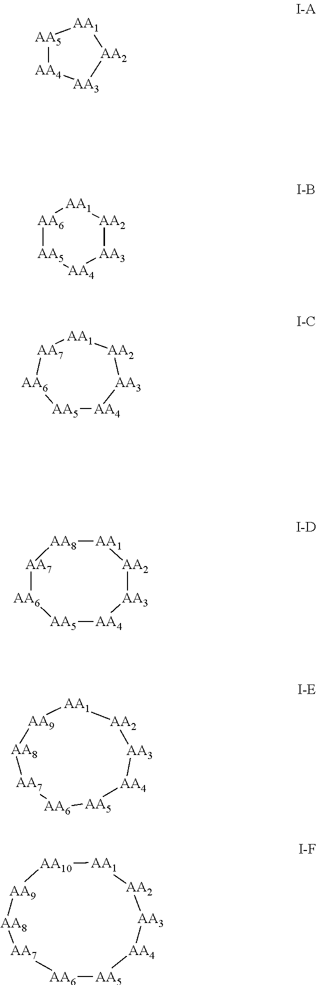

wherein each of AA₁, AA₂, AA₃, AA₄, AA₅, AA₆, AA₇, AA₈, AA₉, and AA₁₀, when present, are independently selected from an amino acid; and
wherein:
   at least two of the amino acids are arginine;
   and at least three amino acids are hydrophobic.

In some embodiments, the cyclic peptides disclosed comprise from 6 to 20 amino acids, wherein at least three of the amino acids are arginine and at least three of the amino acids have a hydrophobic side chain selected from an aryl or heteroaryl, wherein the aryl and heteroaryl are optionally substituted. In some embodiments, the cyclic peptides comprise at least one hydrophobic amino acid having an aryl side chain and at least one hydrophobic amino acid having a heteroaryl side chain. In some embodiments, the cyclic peptide comprises two hydrophobic amino acids having an aryl side chain and one hydrophobic amino acid having a heteroaryl side chain.

In some embodiments, the cyclic peptide comprises at least three, at least four, at least five, at least six, or at least seven arginines. In some embodiments, the cyclic peptide comprises four arginines. In some embodiments, the cyclic peptide comprises five arginines.

In some embodiments, the cyclic peptide comprises at least three, at least four, or at least five hydrophobic amino acids having an aryl or heteroaryl side chain. In some embodiments, the cyclic peptide comprises at least three, at least four, or at least five consecutive hydrophobic amino acids. In some embodiments, the cyclic peptide comprises at least three consecutive hydrophobic amino acids. In some embodiments, at least two, at least three, at least four, or at least five consecutive hydrophobic amino acids have alternating chirality. In some embodiments, at least two, at least three, at least four, or at least five consecutive hydrophobic amino acids have the same chirality.

In some embodiments, the cyclic peptide has a structure of:

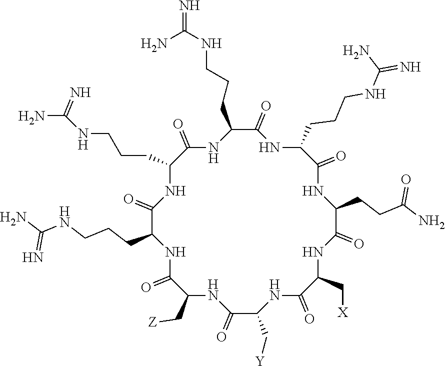

wherein each of X, Y, and Z are a hydrophobic amino acid.

In some embodiments, the cyclic peptide has a structure of:

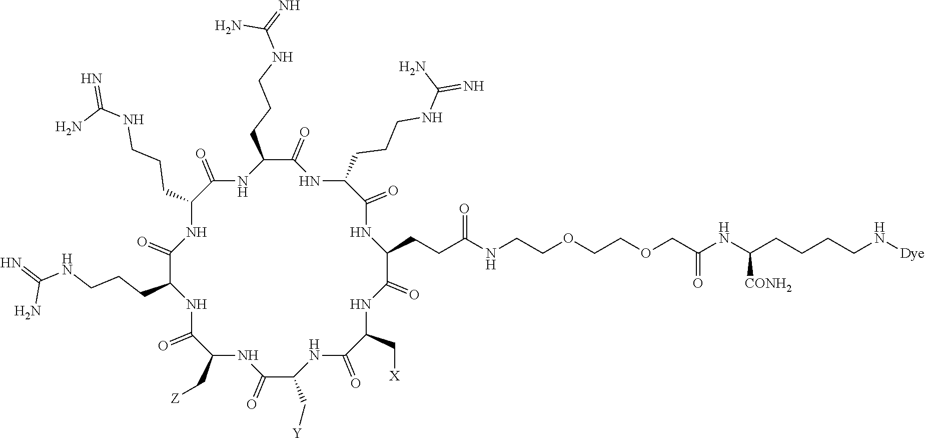

wherein each of X, Y, and Z are a hydrophobic amino acid.

### Hydrophobic Amino Acids

In some embodiments, the amino acid having a hydrophobic side chain is independently an amino acid having a hydrophobic aromatic side chain. In some embodiments, the aromatic side chain is aryl. In some embodiments, the hydrophobic side chain is heteroaryl. In some embodiments, an amino acid having a hydrophobic aromatic side chain is naphthylalanine, phenylglycine, homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. The structures of a few of these non-natural aromatic hydrophobic amino acids (prior to incorporation into the peptides disclosed herein) are provided below. In particular embodiments, the hydrophobic amino acid is piperidine-2-carboxylate, naphthylalanine, tryptophan, 3-(3-benzothienyl)-alanine, or phenylalanine, each of which is optionally substituted with one or more substituents.

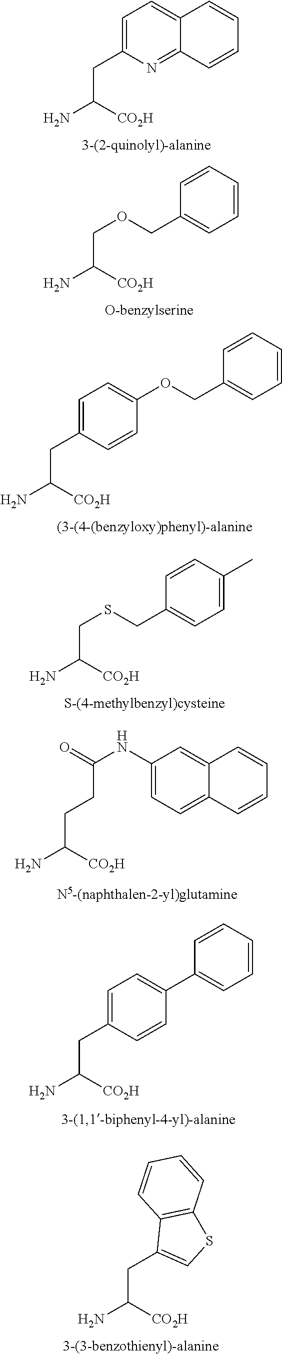

In some embodiments, the cyclic peptide comprises a hydrophobic amino acid selected from the group consisting of L-3-benzothienylalanine, L-4-fluorophenylalanine, D-4-fluorophenylalanine, L-1-naphthylalanine, L-2-naphthylalanine, L-2-pyridylalanine, D-2-pyridylalanine, L-4-pyridylalanine, D-4-pyridylalanine, L-phenylalanine, D-phenylalanine, and combinations thereof. In some embodiments, the cyclic peptide comprises the hydrophobic amino acids L-phenylalanine, D-phenylalanine, and L-2-napthylalanine. In some embodiments, the cyclic peptide comprises the hydrophobic amino acids L-phenylalanine, D-phenylalanine, and L-3-benzothienylalanine. In some embodiments, the cyclic peptide comprises the hydrophobic amino acids L-phenylalanine, D-4-pyridylalanine, and L-2-napthylalanine.

In some embodiments, each amino acid having a hydrophobic side chain is independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylate, 3-(3-benzothienyl)-alanine, or norleucine, each of which is optionally substituted with one or more substituents.

In some embodiments, the amino acid having a hydrophobic side chain is independently an amino acid having a hydrophobic non-aromatic side chain. In some embodiments, an amino acid having a hydrophobic non-aromatic side chain is glycine, alanine, valine, leucine, isoleucine, methionine, or proline. In other embodiments, the amino acid having a hydrophobic non-aromatic side chain has a side chain comprising a C₅-C₄₀ alkyl, alkenyl, alkynyl, acyl, alkylcarboxamidyl, alkoxycarbonyl, carbocyclyl, or heterocyclyl.

Those skilled in the art will appreciate that the N- and/or C-termini of the above non-natural aromatic hydrophobic amino acids, upon incorporation into the peptides disclosed herein, form amide bonds.

The optional substituent can be any atom or group which does not significantly reduce the cytosolic delivery efficiency of the cCPP, e.g., a substituent that does not reduce the relative cytosolic delivery efficiency to less than that of c (FΦRRRRQ). In some embodiments, the optional substituent can be a hydrophobic substituent or a hydrophilic substituent. In certain embodiments, the optional substituent is a hydrophobic substituent. In some embodiments, the substituent increases the solvent-accessible surface area (as defined herein below) of the hydrophobic amino acid. In some embodiments, the substituent can be a halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. In some embodiments, the substituent is a halogen.

Amino acids having higher hydrophobicity values can be selected to improve cytosolic delivery efficiency of a cCPP relative to amino acids having a lower hydrophobicity value. In some embodiments, each hydrophobic amino acid independently has a hydrophobicity value which is greater than that of glycine. In other embodiments, each hydrophobic amino acid independently is a hydrophobic amino acid having a hydrophobicity value which is greater than that of alanine. In still other embodiments, each hydrophobic amino acid independently has a hydrophobicity value which is greater or equal to that of phenylalanine. Hydrophobicity may be measured using hydrophobicity scales known in the art. Table 2 below lists hydrophobicity values for various amino acids as reported by Eisenberg and Weiss (Proc. Natl. Acad. Sci. U.S.A 1984; 81 (1): 140-144), Engleman, et al. (Ann. Rev. of Biophys. Biophys. Chem.. 1986; 1986 (15): 321-53), Kyte and Doolittle (J. Mol. Biol. 1982; 157 (1): 105-132), Hoop and Woods (Proc. Natl. Acad. Sci. U.S.A. 1981; 78 (6): 3824-3828), and Janin (Nature. 1979; 277 (5696): 491-492), the entirety of each of which is herein incorporated by reference in its entirety. In particular embodiments, hydrophobicity is measured using the hydrophobicity scale reported in Engleman, et al.

TABLE 2

| | | Eisenberg | | Kyrie | Hoop | |
|---|---|---|---|---|---|---|
| Amino | | and | Engleman | and | and | |
| Acid | Group | Weiss | et al. | Doolittle | Woods | Janin |
| Ile | Nonpolar | 0.73 | 3.1 | 4.5 | −1.8 | 0.7 |
| Phe | Nonpolar | 0.61 | 3.7 | 2.8 | −2.5 | 0.5 |
| Val | Nonpolar | 0.54 | 2.6 | 4.2 | −1.5 | 0.6 |
| Leu | Nonpolar | 0.53 | 2.8 | 3.8 | −1.8 | 0.5 |
| Trp | Nonpolar | 0.37 | 1.9 | −0.9 | −3.4 | 0.3 |
| Met | Nonpolar | 0.26 | 3.4 | 1.9 | −1.3 | 0.4 |
| Ala | Nonpolar | 0.25 | 1.6 | 1.8 | −0.5 | 0.3 |
| Gly | Nonpolar | 0.16 | 1.0 | −0.4 | 0.0 | 0.3 |
| Cys | Unch/Polar | 0.04 | 2.0 | 2.5 | −1.0 | 0.9 |
| Tyr | Unch/Polar | 0.02 | −0.7 | −1.3 | −2.3 | −0.4 |
| Pro | Nonpolar | −0.07 | −0.2 | −1.6 | 0.0 | −0.3 |
| Thr | Unch/Polar | −0.18 | 1.2 | −0.7 | −0.4 | −0.2 |
| Ser | Unch/Polar | −0.26 | 0.6 | −0.8 | 0.3 | −0.1 |
| His | Charged | −0.40 | −3.0 | −3.2 | −0.5 | −0.1 |
| Glu | Charged | −0.62 | −8.2 | −3.5 | 3.0 | −0.7 |
| Asn | Unch/Polar | −0.64 | −4.8 | −3.5 | 0.2 | −0.5 |
| Gln | Unch/Polar | −0.69 | −4.1 | −3.5 | 0.2 | −0.7 |
| Asp | Charged | −0.72 | −9.2 | −3.5 | 3.0 | −0.6 |
| Lys | Charged | −1.10 | −8.8 | −3.9 | 3.0 | −1.8 |
| Arg | Charged | −1.80 | −12.3 | −4.5 | 3.0 | −1.4 |

In some embodiments, an arginine is adjacent to a hydrophobic amino acid. In some embodiments, the arginine has the same chirality as the hydrophobic amino acid. In some embodiments, at least two arginines are adjacent to each other. In other embodiments, three arginines are adjacent to each other. In some embodiments, at least two hydrophobic amino acids are adjacent to each other. In other embodiments, at least three hydrophobic amino acids are adjacent to each other. In other embodiments, the CPPs described herein comprise at least two consecutive hydrophobic amino acids and at least two adjacent arginines. In further embodiments, one hydrophobic amino acid is adjacent to one of the arginines. In still other embodiments, the CPPs described herein comprise at least three adjacent hydrophobic amino acids and at least three adjacent arginines. In further embodiments, one hydrophobic amino acid is adjacent to one of the arginines. These various combinations of amino acids can have any arrangement of D and L amino acids, e.g., any of the sequences described in the preceding paragraph.

In some embodiments, any four adjacent amino acids in the cCPPs described herein (e.g., cCPPs of Formulae IA-IF and IVA-IVF) can have one of the following sequences: AA_{H1}-R—R—R or R—R—R-AA_{H1}, wherein each AA_{H1} is independently an amino acid having a hydrophobic side chain.

In some embodiments, each hydrophobic amino acid is independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylate, or norleucine, each of which is optionally substituted with one or more substituents.

In some embodiments, the amino acid having a hydrophobic side chain is independently an amino acid having a hydrophobic non-aromatic side chain. In some embodiments, an amino acid having a hydrophobic non-aromatic side chain is glycine, alanine, valine, leucine, isoleucine, methionine, or proline. In other embodiments, the amino acid having a hydrophobic non-aromatic side chain is a non-natural amino acid and has a side chain comprising a C₅-C₄₀ alkyl, alkenyl, alkynyl, acyl, alkylcarboxamidyl, alkoxycarbonyl, carbocyclyl, or heterocyclyl.

In some embodiments, the amino acid having a hydrophobic side chain is independently an amino acid having a hydrophobic aromatic side chain. In some embodiments, an amino acid having a hydrophobic aromatic side chain is naphthylalanine, phenylglycine, homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. The structures of a few of these non-natural aromatic hydrophobic amino acids (prior to incorporation into the peptides disclosed herein) are provided below. In particular embodiments, the hydrophobic amino acid is piperidine-2-carboxylate, naphthylalanine, tryptophan, 3-(3-benzothienyl)-alanine, or phenylalanine, each of which is optionally substituted with one or more substituents.

In some embodiments, the hydrophobic amino acid has a hydrophobicity value which is greater than that of glycine. In other embodiments, the hydrophobic amino acid has a hydrophobicity value which is greater than that of alanine. In still other embodiments, the hydrophobic amino acid has a hydrophobicity value which is greater than that of phenylalanine, e.g., as measured using the hydrophobicity scales described above, including Eisenberg and Weiss (Proc. Natl. Acad. Sci. U.S.A 1984; 81 (1): 140-144), Engleman, et al. (Ann. Rev. of Biophys. Biophys. Chem.. 1986; 1986 (15): 321-53), Kyte and Doolittle (J. Mol. Biol. 1982; 157 (1): 105-132), Hoop and Woods (Proc. Natl. Acad. Sci. U.S.A. 1981; 78 (6): 3824-3828), and Janin (Nature. 1979; 277 (5696): 491-492), (see Table 2 above). In particular embodiments, hydrophobicity is measured using the hydrophobicity scale reported in Engleman, et al.

The presence of a hydrophobic amino acid on the N- or C-terminus of a D-Arg or L-Arg, or a combination thereof, has also been found to improve the cytosolic uptake of the CPP (and the attached cargo). For example, in some embodiments, the CPPs disclosed herein may include AA_{H1}-D-Arg or D-Arg-AA_{H1}. In other embodiments, the CPPs disclosed herein may include AA_{H1}-L-Arg or L-Arg-AA_{H1}. In some embodiments, the presence of the hydrophobic amino acid on the N- or C-terminus of the D-Arg or L-Arg, or a combination thereof, in the CPP improves the cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween. In some embodiments, the presence of the hydrophobic amino acid on the N- and/or C-terminus of the D-Arg and/or L-Arg in the CPP improves the cytosolic uptake efficiency by about 20 fold.

The size of the hydrophobic amino acid on the N- or C-terminus of the D-Arg or an L-Arg, or a combination thereof, may be selected to improve cytosolic delivery efficiency of the CPP. For example, a larger hydrophobic amino acid on the N- or C-terminus of a D-Arg or L-Arg, or a combination thereof, improves cytosolic delivery efficiency compared to an otherwise identical sequence having a smaller hydrophobic amino acid. The size of the hydrophobic amino acid can be measured in terms of molecular weight of the hydrophobic amino acid, the steric effects of the hydrophobic amino acid, the solvent-accessible surface area (SASA) of the side chain, or combinations thereof. In some embodiments, the size of the hydrophobic amino acid is measured in terms of the molecular weight of the hydrophobic amino acid, and the larger hydrophobic amino acid has a side chain with a molecular weight of at least about 90 g/mol, or at least about 130 g/mol, or at least about 141 g/mol. In particular embodiments, the size of the amino acid is measured in terms of the SASA of the hydrophobic side chain, and the larger hydrophobic amino acid has a side chain with a SASA greater than that of alanine, or greater than that of glycine. In other embodiments, AA_{H1} has a hydrophobic side chain with a SASA greater than or equal to about piperidine-2-carboxylate, greater than or equal to about tryptophan, greater than or equal to about phenylalanine, or equal to or greater than about naphthylalanine. In some embodiments, AA_{H1} and AA_{H2} independently have a side with a SASA in the range of from about 200 Å² to about 1000 Å², e.g, about 250 Å², 300 Å², 350 Å², 400 Å², 450 Å², 500 Å², 550 Å², 650 Å², 700 Å², 750 Å², 800 Å², 850 Å², 900 Å², and about 950 Å², inclusive of all values and subranges therebetween.

In some embodiments, a hydrophobic amino acid has a side chain with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². In some embodiments, a hydrophobic amino acid has a side chain side with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². In some embodiments, the side chains of a first hydrophobic amino acid and a second hydrophobic amino acid have a combined SASA of at least about 350 Å², at least about 360 Å², at least about 370 Å², at least about 380 Å², at least about 390 Å², at least about 400 Å², at least about 410 Å², at least about 420 Å², at least about 430 Å², at least about 440 Å², at least about 450 Å², at least about 460 Å², at least about 470 Å², at least about 480 Å², at least about 490 Å², greater than about 500 Å², at least about 510 Å², at least about 520 Å², at least about 530 Å², at least about 540 Å², at least about 550 Å², at least about 560 Å², at least about 570 Å², at least about 580 Å², at least about 590 Å², at least about 600 Å², at least about 610 Å², at least about 620 Å², at least about 630 Å², at least about 640 Å², greater than about 650 Å², at least about 660 Å², at least about 670 Å², at least about 680 Å², at least about 690 Å², or at least about 700 Å². In some embodiments, the second hydrophobic amino acid has a side chain with a SASA that is less than or equal to the SASA of the hydrophobic side chain of the first hydrophobic amino acid. By way of example, and not by limitation, a CPP having a Nal-Arg motif exhibits improved cytosolic delivery efficiency compared to an otherwise identical CPP having a Phe-Arg motif; a CPP having a Phe-Nal-Arg motif exhibits improved cytosolic delivery efficiency compared to an otherwise identical CPP having a Nal-Phe-Arg motif; and a phe-Nal-Arg motif exhibits improved cytosolic delivery efficiency compared to an otherwise identical CPP having a nal-Phe-Arg motif. In some embodiments, the presence of the larger hydrophobic amino acid on the N- or C-terminus of the D-Arg or L-Arg, or a combination thereof, in the CPP improves cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween. In particular embodiments, the presence of the larger hydrophobic amino acid on the N- and/or C-terminus of the D-Arg and/or L-Arg in the CPP improves the cytosolic uptake efficiency by about 20 fold.

As used herein, “hydrophobic surface area” or “SASA” refers to the surface area (reported as square Ångstroms; Å²) of an amino acid side chain that is accessible to a solvent. In particular embodiments, SASA is calculated using the ‘rolling ball’ algorithm developed by Shrake & Rupley (J Mol Biol. 79 (2): 351-71), which is herein incorporated by reference in its entirety for all purposes. This algorithm uses a “sphere” of solvent of a particular radius to probe the surface of the molecule. A typical value of the sphere is 1.4 Å, which approximates to the radius of a water molecule.

SASA values for certain side chains are shown below in Table 3. In certain embodiments, the SASA values described herein are based on the theoretical values listed in Table 3 below, as reported by Tien, et al. (PLOS ONE 8(11): e80635. https://doi.org/10.1371/journal.pone.0080635), which is herein incorporated by reference in its entirety for all purposes.

TABLE 3

| | | | Miller et al. | Rose et al. |
|---|---|---|---|---|
| Residue | Theoretical | Empirical | (1987) | (1985) |
| Alanine | 129.0 | 121.0 | 113.0 | 118.1 |
| Arginine | 274.0 | 265.0 | 241.0 | 256.0 |
| Asparagine | 195.0 | 187.0 | 158.0 | 165.5 |
| Aspartate | 193.0 | 187.0 | 151.0 | 158.7 |
| Cysteine | 167.0 | 148.0 | 140.0 | 146.1 |
| Glutamate | 223.0 | 214.0 | 183.0 | 186.2 |
| Glutamine | 225.0 | 214.0 | 189.0 | 193.2 |
| Glycine | 104.0 | 97.0 | 85.0 | 88.1 |
| Histidine | 224.0 | 216.0 | 194.0 | 202.5 |
| Isoleucine | 197.0 | 195.0 | 182.0 | 181.0 |
| Leucine | 201.0 | 191.0 | 180.0 | 193.1 |
| Lysine | 236.0 | 230.0 | 211.0 | 225.8 |
| Methionine | 224.0 | 203.0 | 204.0 | 203.4 |
| Phenylalanine | 240.0 | 228.0 | 218.0 | 222.8 |
| Proline | 159.0 | 154.0 | 143.0 | 146.8 |
| Serine | 155.0 | 143.0 | 122.0 | 129.8 |
| Threonine | 172.0 | 163.0 | 146.0 | 152.5 |
| Tryptophan | 285.0 | 264.0 | 259.0 | 266.3 |
| Tyrosine | 263.0 | 255.0 | 229.0 | 236.8 |
| Valine | 174.0 | 165.0 | 160.0 | 164.5 |

The chirality of the amino acids (i.e., D or L amino acids) can be selected to improve cytosolic delivery efficiency of the CPP (and the attached cargo as described below). In some embodiments, the hydrophobic amino acid on the N- or C-terminus of an arginine has the same or opposite chirality as the adjacent arginine. In some embodiments, a hydrophobic amino acid has the same chirality as an adjacent arginine. For example, when the arginine is D-arg (i.e. “r”), the hydrophobic amino acid is a D-amino acid, and when the arginine is L-Arg (i.e., “R”), the hydrophobic amino acid is an L-amino acid. Accordingly, in some embodiments, the CPPs disclosed herein may include at least one of the following motifs: D-hydrophobic amino acid-D-arg, D-arg-D-hydrophobic amino acid, L-hydrophobic amino acid-L-Arg, or L-Arg-L-hydrophobic amino acid. In some embodiments, the presence of the hydrophobic amino acid having the same chirality as the adjacent arginine improves cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold inclusive of all values and subranges therebetween. In some embodiments, the presence of the hydrophobic amino acid having the same chirality as the adjacent arginine improves the cytosolic uptake efficiency by about 2.5 fold.

As discussed above, the disclosure provides for various modifications to a cyclic peptide sequence which may improve cytosolic delivery efficiency. In some embodiments, improved cytosolic uptake efficiency can be measured by comparing the cytosolic delivery efficiency of the CPP having the modified sequence to a proper control sequence. In some embodiments, the control sequence does not include a particular modification (e.g., matching chirality of R and the hydrophobic amino acid) but is otherwise identical to the modified sequence. In other embodiments, the control has the following sequence: cyclo(FΦRRRRQ) or cyclo(FfΦRrRrQ). In particular embodiments, the control is cyclo(FfΦRrRrQ).

As used herein cytosolic delivery efficiency refers to the ability of a CPP to traverse a cell membrane and enter the cytosol. In some embodiments, cytosolic delivery efficiency of the CPP is not dependent on a receptor or a cell type. Cytosolic delivery efficiency can refer to absolute cytosolic delivery efficiency or relative cytosolic delivery efficiency. The terms “cytosolic delivery efficiency” and “cytosolic entry efficiency” are used interchangeably herein.

Absolute cytosolic delivery efficiency is the ratio of cytosolic concentration of a cCPP (or a cCPP-cargo conjugate) over the concentration of the cCPP (or the cCPP-cargo conjugate) in the growth medium. Relative cytosolic delivery efficiency refers to the concentration of a cCPP in the cytosol compared to the concentration of a control cCPP in the cytosol. Quantification can be achieved by fluorescently labeling the cCPP (e.g., with a FITC dye or naphthofluorescein (NF)) and measuring the fluorescence intensity using techniques well-known in the art. In some embodiments, absolute cytosolic delivery efficiency is measured in the presence of serum, for example, fetal bovine serum (FBS). In some embodiments, FBS is present in an amount from about 0.5% to about 15%, for example, about 0.5%, about 1%, about 2%, about 3%, about 4%, about 5%, about 6%, about 7%, about 8%, about 9%, about 10%, about 11%, about 12%, about 13%, about 14%, or about 15%. In some embodiments, absolute cytosolic delivery efficiency is measured in the presence of 1% FBS. In some embodiments, absolute cytosolic delivery efficiency is measured in the presence of 10% FBS.

In some embodiments, relative cytosolic delivery efficiency is determined by comparing (i) the amount of a cCPP of the invention internalized by a cell type (e.g., HeLa cells) to (ii) the amount of the control cCPP internalized by the same cell type. To measure relative cytosolic delivery efficiency, the cell type may be incubated in the presence of a cCPP of the invention for a specified period of time (e.g., 30 minutes, 1 hour, 2 hours, etc.) after which the amount of the cCPP internalized by the cell is quantified using methods known in the art, e.g., fluorescence microscopy. Separately, the same concentration of the control cCPP is incubated in the presence of the cell type over the same period of time, and the amount of the control cCPP internalized by the cell is quantified. In some embodiments, relative cytosolic delivery efficiency is measured in the presence of serum, for example, fetal bovine serum (FBS). In some embodiments, FBS is present in an amount from about 0.5% to about 15%, for example, about 0.5%, about 1%, about 2%, about 3%, about 4%, about 5%, about 6%, about 7%, about 8%, about 9%, about 10%, about 11%, about 12%, about 13%, about 14%, or about 15%. In some embodiments, relative cytosolic delivery efficiency is measured in the presence of 1% FBS. In some embodiments, relative cytosolic delivery efficiency is measured in the presence of 10% FBS.

In some embodiments, relative cytosolic delivery efficiency can be determined by measuring the IC₅₀ of a cCPP having a modified sequence for an intracellular target, and comparing the IC₅₀ of the cCPP having the modified sequence to a proper control sequence (as described herein).

In some embodiments, the relative cytosolic delivery efficiency of the CPPs described herein is in the range of from about 50% to about 450% compared to cyclo(FfΦRrRrQ), e.g., about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 510%, about 520%, about 530%, about 540%, about 550%, about 560%, about 570%, about 580%, or about 590%, inclusive of all values and subranges therebetween. In other embodiments, the relative cytosolic delivery efficiency of the CPPs described herein is improved by greater than about 600% compared to cyclo(FΦRRRRQ) or cyclo(FfΦRrRrQ). In some embodiments, the cytosolic delivery efficiency is about 375% of that of cyclo(FΦRRRRQ) or cyclo(FfΦRrRrQ).

In some embodiments, the cytosolic delivery efficiency is about 115% of that of cyclo(FΦRRRRQ) or cyclo(FfΦRrRrQ).

In other embodiments, the absolute cytosolic delivery efficacy of from about 40% to about 100%, e.g., about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, inclusive of all values and subranges therebetween.

In some embodiments, the disclosure provides for various modifications to a cyclic peptide sequence which may improve total cellular uptake. In some embodiments, improved total cellular uptake can be measured by comparing the total cellular uptake of the CPP having the modified sequence to a proper control sequence. In some embodiments, the control sequence does not include a particular modification described herein but is otherwise identical to the modified sequence. In other embodiments, the control has the following sequence: cyclo(FΦRRRRQ) or cyclo(FfΦRrRrQ). In particular embodiments, the control is cyclo(FfΦRrRrQ).

As used herein total cellular uptake refers to the amount of a CPP that enters a cell (e.g., the sum of the amount of CPP in the cytosol and CPP in the endosome). In some embodiments, the total cellular uptake of a CPP of the disclosure is improved by between about 5% and about 400%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, or about 400%, inclusive of all values and subranges therebetween, as compared to a control CPP. In some embodiments, the total cellular uptake of a CPP of the disclosure is from about 75% to about 100%, for example about 75% to 90%, about 80% to 90%, about 90% to about 100%, about 85% to about 100%, of the total cellular uptake of a control CPP, for example, cyclo(FfΦRrRrQ). In some embodiments, the total cellular uptake of a CPP of the disclosure is at least about 75%, at least about 80%, at least about 85%, at least about 90%, at least about 95%, at least about 96%, at least about 97%, at least about 98%, at least 99%, or at least 100% of the total cellular uptake of a control CPP, for example, cyclo(FfΦRrRrQ).

In some embodiments, the disclosure provides for various modifications to a cyclic peptide sequence which may improve endosomal escape efficiency, γ. In some embodiments, improved endosomal escape efficiency can be measured by comparing the endosomal escape efficiency of the CPP having the modified sequence to a proper control sequence. In some embodiments, the control sequence does not include a particular modification (e.g., matching chirality of R and the hydrophobic amino acid) but is otherwise identical to the modified sequence. In other embodiments, the control has the following sequence: cyclic(FΦRRRRQ) or cyclo(FfΦRrRrQ).

In some embodiments, the endosomal escape efficiency of the CPPs described herein is in the range of from about 50% to about 450% compared to cyclo(FfΦRrRrQ), e.g., about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 510%, about 520%, about 530%, about 540%, about 550%, about 560%, about 570%, about 580%, or about 590%, inclusive of all values and subranges therebetween. In other embodiments, the endosomal escape efficiency of the CPPs described herein is improved compared to cyclo(FΦRRRRQ) by greater than or equal to about 108%. In other embodiments, the endosomal escape efficiency of the CPPs described herein is improved compared to cyclo(FfΦRrRrQ) by greater than or equal to about 138%. In other embodiments, the endosomal escape efficiency of the CPPs described herein is improved compared to cyclo(FfΦRrRrQ) by greater than or equal to about 164%. In other embodiments, the endosomal escape efficiency of the CPPs described herein is improved compared to cyclo(FfΦRrRrQ) by greater than or equal to about 198%.

In some embodiments, the cCPPs disclosed herein (e.g., Formulae I, I-A to I-F, and Formula IV-A to IV-F) are selected from the sequences provided in Table 4 below.

TABLE 4

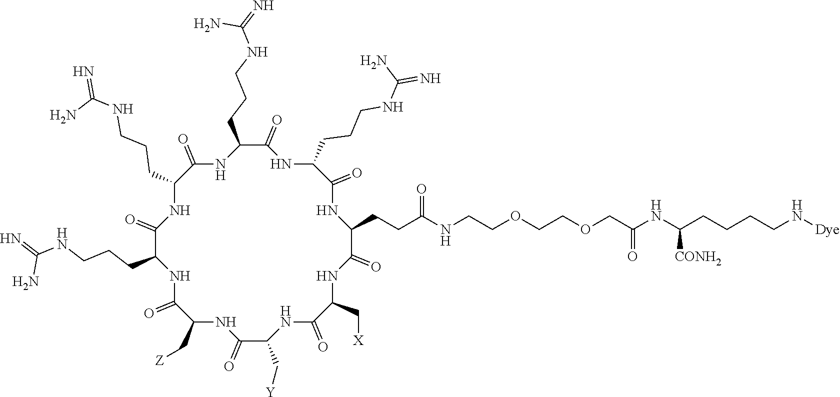

| | | | | Cytosolic Delivery | Cytosolic Delivery |
|---|---|---|---|---|---|
| Peptide | Peptide Sequenceª | | | Efficiency at 10% | Efficiency at 1% |
| No. | X = | Y = | Z = | FBS (MFI^{NF}, %)^{b} | FBS (MFI^{NF}, %)^{b} |
| CPP12 | Phe | D-Phe | 2-Nal | 100 | 100 |
| CPP12-1 | Phe | D-Phe | 1-Nal | 69 ± 9 | 105 ± 16 |
| CPP12-2 | Phe | D-Phe | Bta | 375 ± 48 | 85 ± 7 |
| CPP12-3 | Phe | D-Fpa | 2-Nal | 59 ± 12 | 83 ± 2 |
| CPP12-4 | Phe | D-2-Pya | 2-Nal | 29 ± 9 | 35 ± 5 |
| CPP12-5 | Phe | D-4-Pya | 2-Nal | 115 ± 18 | 36 ± 5 |
| CPP12-6 | Phe | D-Tyr | 2-Nal | 32 ± 15 | 30 ± 12 |
| CPP12-7 | Fpa | D-Phe | 2-Nal | 74 ± 9 | 86 ± 15 |
| CPP12-8 | 2-Pya | D-Phe | 2-Nal | 27 ± 8 | 41 ± 16 |
| CPP12-9 | 4-Pya | D-Phe | 2-Nal | 22 ± 9 | 57 ± 18 |
| CPP12-10 | Tyr | D-Phe | 2-Nal | 25 ± 15 | 44 ± 10 |

^{a}Bta, L-3-benzothienylalanine; Fpa, L-4-fluorophenylalanine; D-Fpa, D-4-fluorophenylalanine; 1-Nal, L-1-naphthylalanine; 2-Nal, L-2-naphthylalanine; 2-Pya, L-2-pyridylalanine; D-2-Pya, D-2-pyridylalanine; 4-Pya, L-4-pyridylalanine; D-4-Pya, D-4-pyridylalanine.
^{b}All values are relative to that of CPP12 (100%) and represent the mean ± SD of three independent experiments.

In some embodiments, the cCPPs disclosed herein are selected from:

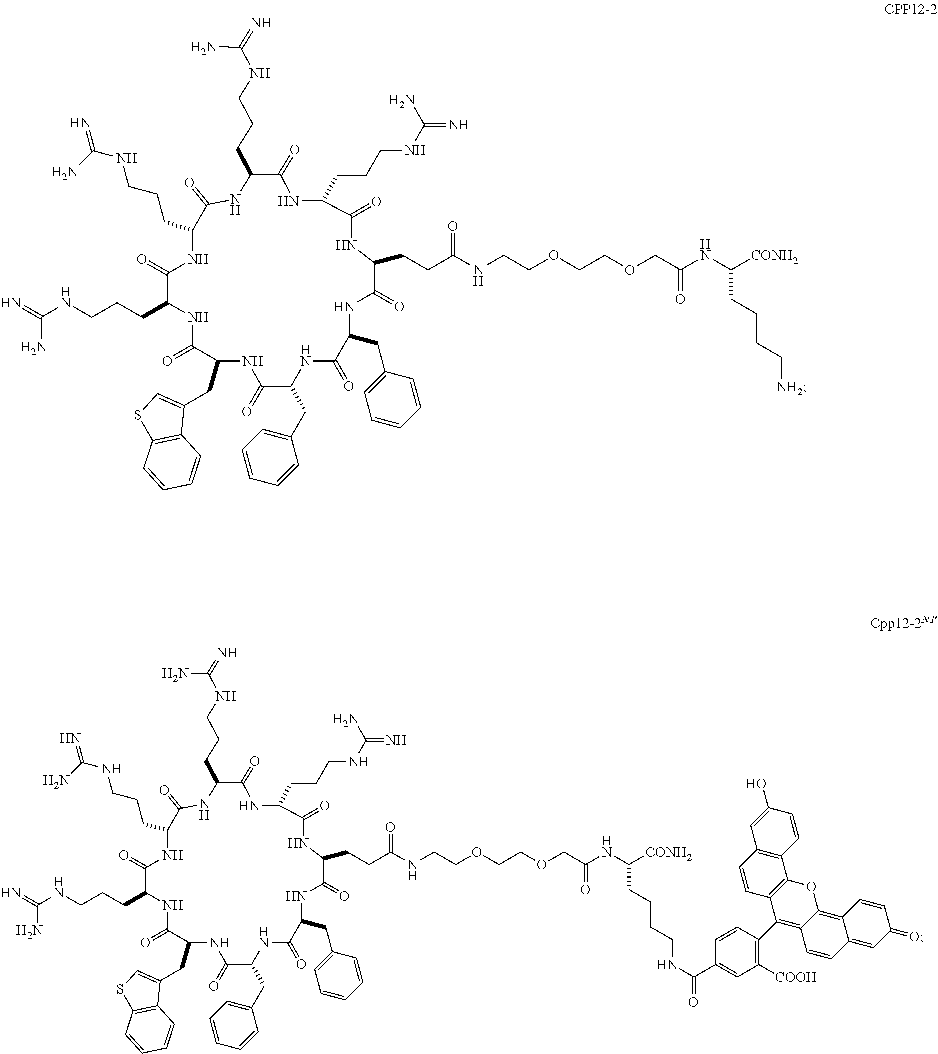

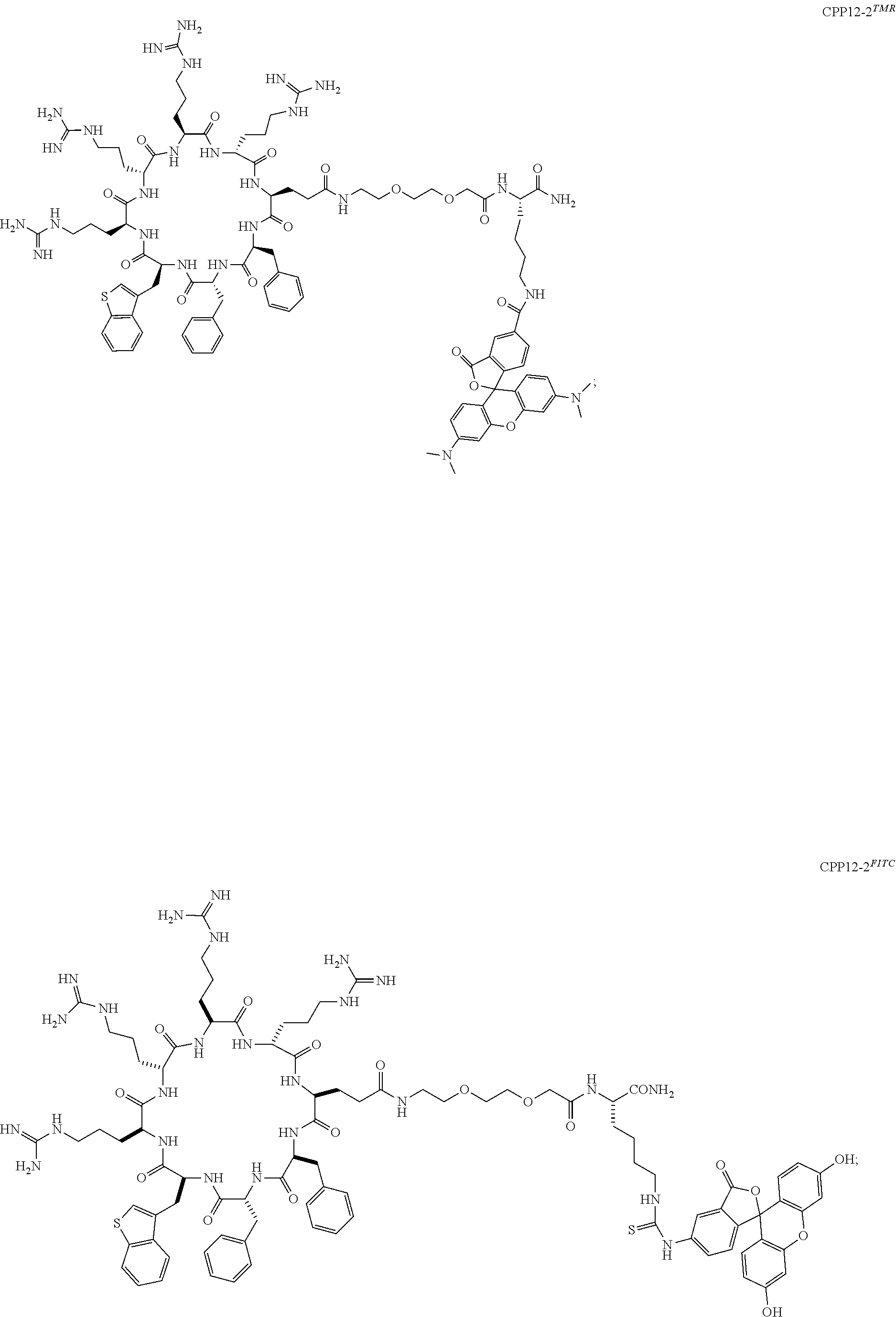

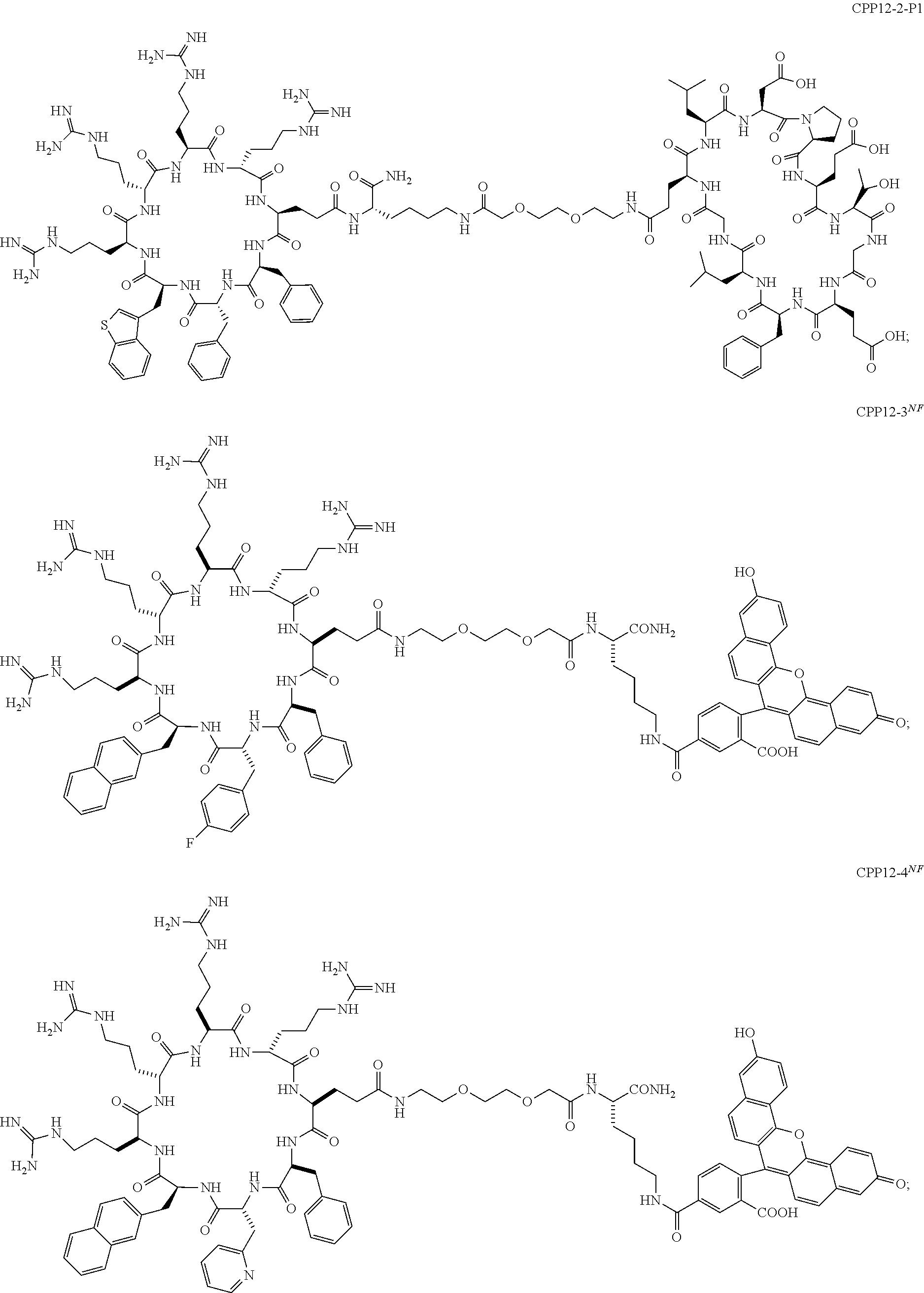

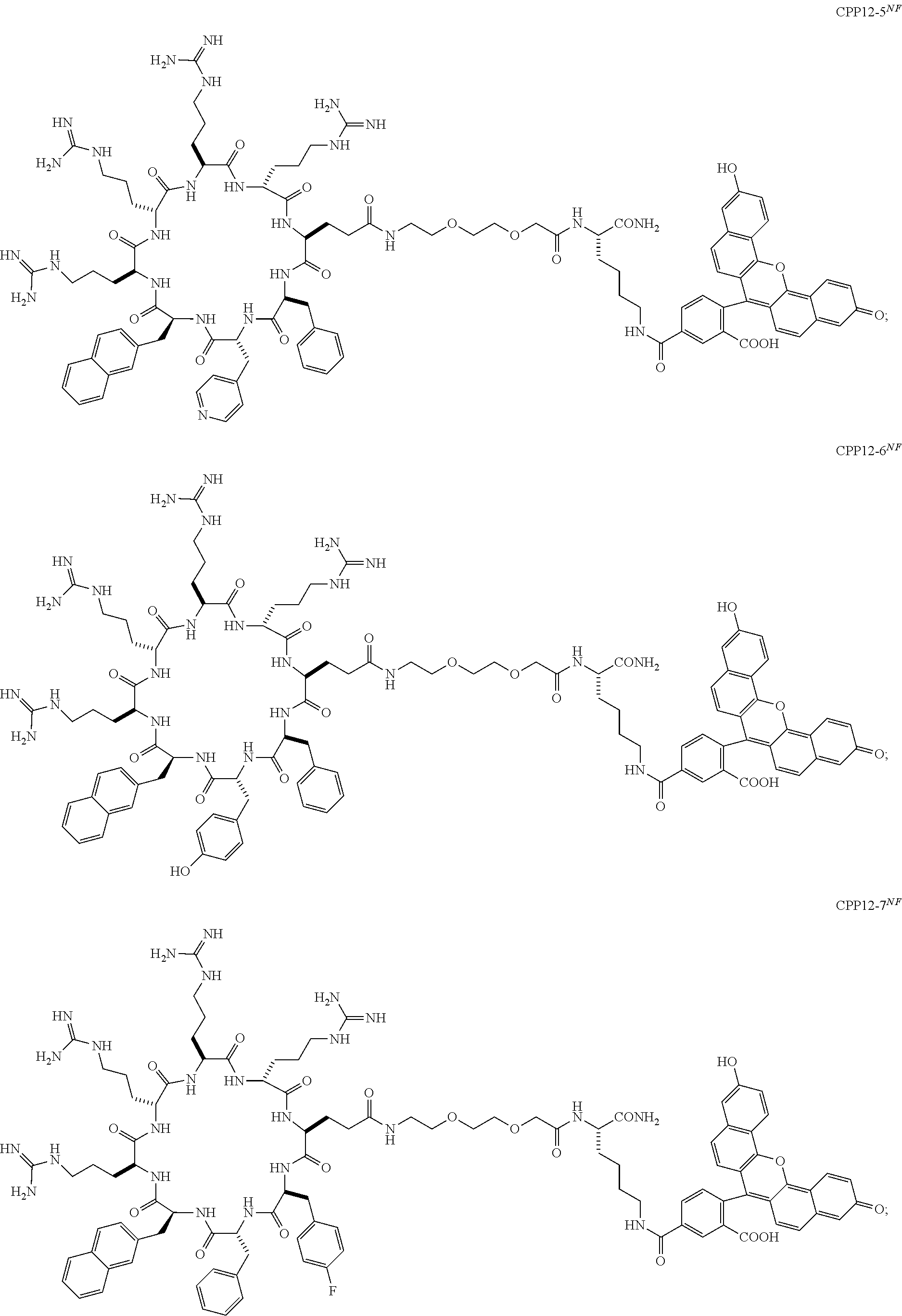

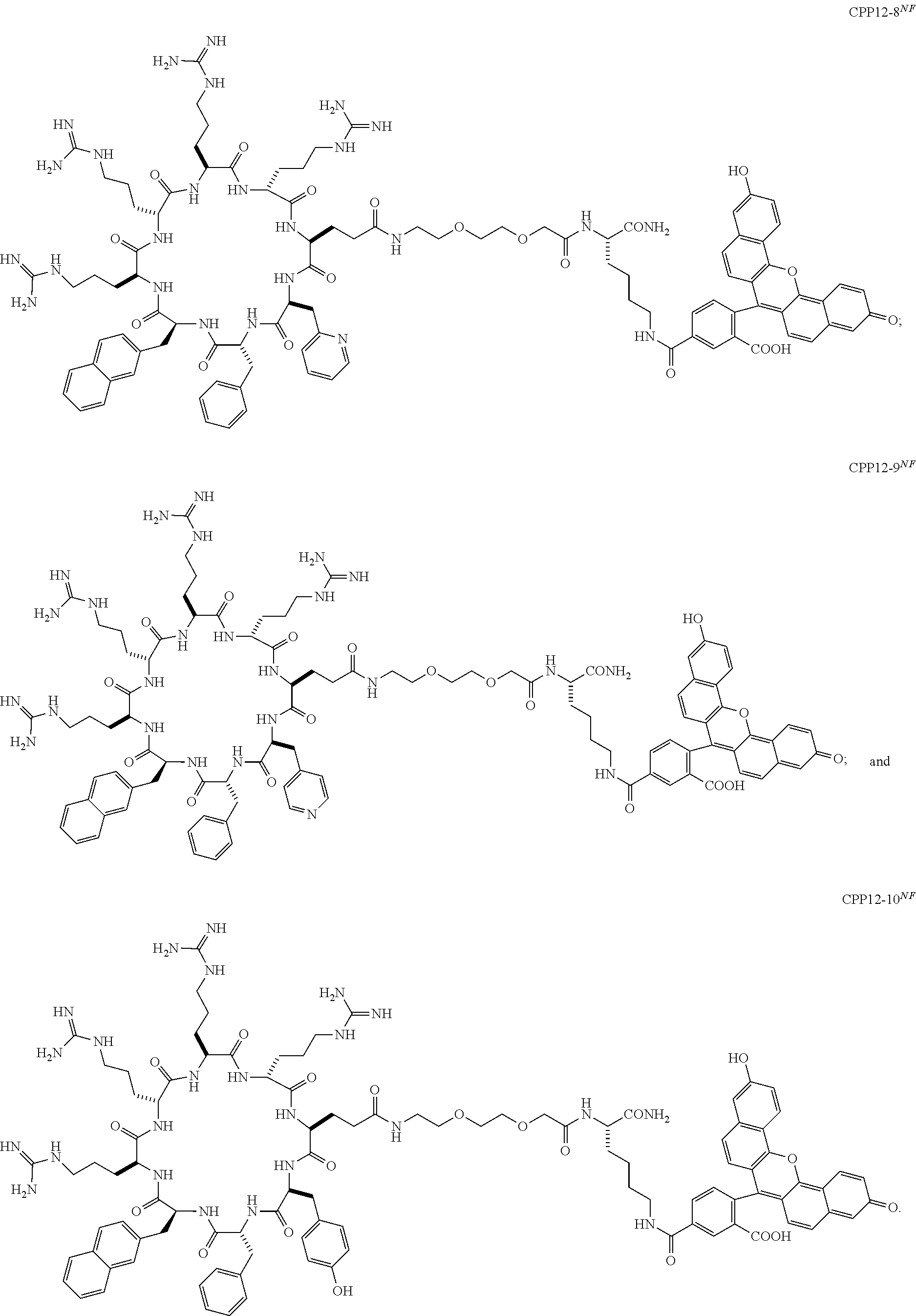

In some embodiments, the cyclic peptide has an amino acid sequence selected from the group consisting of:

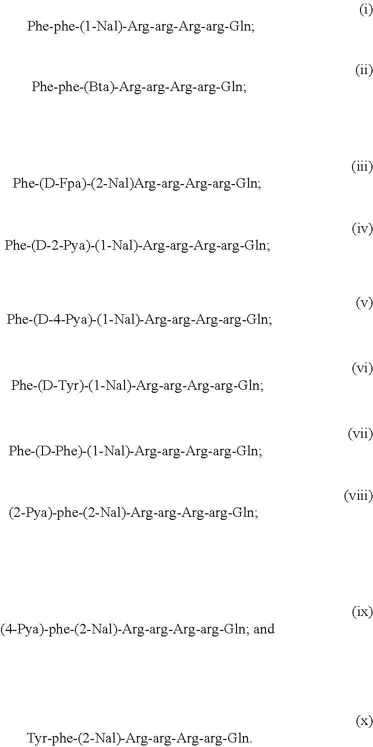

In some embodiments, the cyclic peptide has an amino acid sequence of Phe-phe-(Bta)-Arg-arg-Arg-arg-Gln. In some embodiments, the cyclic peptide has a sequence of Phe-(D-4-Pya)-(2-Nal)-Arg-arg-Arg-arg-Gln.

In some embodiments, the cyclic peptide further comprises an amino acid selected from the group consisting of glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylate, or norleucine, each of which is optionally substituted with one or more substituents.

### Cargo

In some embodiments, the CPPs disclosed herein can further include a cargo moiety, which may comprise a peptide. The cargo moiety can comprise one or more detectable moieties, one or more therapeutic moieties, one or more targeting moieties, or any combination thereof. In some embodiments, the cargo moiety may be a peptide sequence or a non-peptidyl therapeutic agent. In some embodiments, the cargo moiety can be coupled to an amino group (e.g., N-terminus), a carboxylate group (e.g., C-terminus), or a side chain of one or more amino acids in the cCPP. In some embodiments, the cCPP and the cargo moiety together are cyclic (referred to herein as “endocyclic”). In the endocyclic system, the cargo moiety may be located between amino acids of the cCPP. In some embodiments, the cargo moiety independently forms a peptide bond with each of the adjacent amino acids. In some embodiments, the cCPP is cyclic and the cargo moiety is appended to the cyclic cell penetrating peptide moiety structure (referred to herein as “exocyclic”). In some embodiments, the cargo moiety is cyclic and the cCPP is cyclic, and together they form a bicyclic system (referred to herein as “bicyclic”).

In some embodiments, the cargo is attached to the cCPP via a linker (“L”). In some embodiments, L is 1 to 22 carbon atoms in length, wherein one or more carbon atoms are each optionally and independently replaced by a group selected from C(O), O, N(O), N(alkyl), S, C₂-alkenyl, C₂-alkynyl, cycloalkyl, aryl, heterocyclyl, and heteroaryl.

In some embodiments, the cCPP further comprises a linker group (“L”), and the cargo is attached to the linker group, forming a bicyclic cCPP and cargo moiety. In certain embodiments, the cCPP further comprises a linker group (“L”), and cargo is attached to the linker group and a side chain of an amino acid of the CPP, forming a bicyclic cCPP and cargo moiety. Examples of linkers used to form a bicyclic include trimesic acid or nitrilotriacetic acid, as described in U.S. Patent App. Pub. 2016/0115202, which is herein incorporated by reference in its entirety for all purposes.

It is also disclosed herein that for the endocyclic structure, some amino acids in the CPP can also be part of the cargo moity. For example, a peptide pentrating moiety FNalRR can be formed from FNal and a cargo moiety comprising two Args. In this case, the two Arg residues perform dual functions. Thus, in some cases the sequence of the cargo moiety is taken into account when referring to the peptide penetrating moiety.

### Cargo Moiety

The cargo moiety can comprise any cargo of interest, for example a linker moiety, a detectable moiety, a therapeutic moiety, a targeting moiety, and the like, or any combination thereof. In some examples, the cargo moiety can comprise one or more additional amino acids (e.g., K, UK, TRV); a linker (e.g., bifunctional linker LC-SMCC); coenzyme A; phosphocoumaryl amino propionic acid (pCAP); 8-amino-3,6-dioxaoctanoic acid (miniPEG); L-2,3-diaminopropionic acid (Dap or J); L-β-naphthylalanine; L-pipecolic acid (Pip); sarcosine; trimesic acid; 7-amino-4-methylcourmarin (Amc); fluorescein isothiocyanate (FITC); L-2-naphthylalanine; norleucine; 2-aminobutyric acid; Rhodamine B (Rho); Dexamethasone (DEX); or combinations thereof.

In some examples the cargo moiety can comprise any of those listed in Table 5, or derivatives or combinations thereof.

TABLE 5
Example cargo moieties

| SEQ | | |
|---|---|---|
| ID NO | Abbreviation | Sequence* |
| 1 | R₅ | RRRRR |
| 2 | A₅ | AAAAA |
| 3 | F₄ | FFFF |
| | PCP | DE(pCAP)LI |
| 4 | A₇ | AAAAAAA |
| 5 | | RARAR |
| 6 | | DADAD |
| 7 | | DΩUD |
| 8 | | UTRV |
| | | D-pThr-Pip-Nal |

*pCAP, phosphocoumaryl amino propionic acid; Ω, norleucine; U, 2-aminobutyric acid; D-pThr is D-phosphothreonine, Pip is L-piperidine-2-carboxylate.

### Detectable Moiety

The detectable moiety can comprise any detectable label. Examples of suitable detectable labels include, but are not limited to, a UV-Vis label, a near-infrared label, a luminescent group, a phosphorescent group, a magnetic spin resonance label, a photosensitizer, a photocleavable moiety, a chelating center, a heavy atom, a radioactive isotope, a isotope detectable spin resonance label, a paramagnetic moiety, a chromophore, or any combination thereof. In some embodiments, the label is detectable without the addition of further reagents.

In some embodiments, the detectable moiety is a biocompatible detectable moiety, such that the compounds can be suitable for use in a variety of biological applications. “Biocompatible” and “biologically compatible”, as used herein, generally refer to compounds that are, along with any metabolites or degradation products thereof, generally non-toxic to cells and tissues, and which do not cause any significant adverse effects to cells and tissues when cells and tissues are incubated (e.g., cultured) in their presence.

The detectable moiety can contain a luminophore such as a fluorescent label or near-infrared label. Examples of suitable luminophores include, but are not limited to, metal porphyrins; benzoporphyrins; azabenzoporphyrine; napthoporphyrin; phthalocyanine; polycyclic aromatic hydrocarbons such as perylene, perylene diimine, pyrenes; azo dyes; xanthene dyes; boron dipyoromethene, aza-boron dipyoromethene, cyanine dyes, metal-ligand complex such as bipyridine, bipyridyls, phenanthroline, coumarin, and acetylacetonates of ruthenium and iridium; acridine, oxazine derivatives such as benzophenoxazine; aza-annulene, squaraine; 8-hydroxyquinoline, polymethines, luminescent producing nanoparticle, such as quantum dots, nanocrystals; carbostyril; terbium complex; inorganic phosphor; ionophore such as crown ethers affiliated or derivatized dyes; or combinations thereof. Specific examples of suitable luminophores include, but are not limited to, Pd (II) octaethylporphyrin; Pt (II)-octaethylporphyrin; Pd (II) tetraphenylporphyrin; Pt (II) tetraphenylporphyrin; Pd (II) meso-tetraphenylporphyrin tetrabenzoporphine; Pt (II) meso-tetrapheny metrylbenzoporphyrin; Pd (II) octaethylporphyrin ketone; Pt (II) octaethylporphyrin ketone; Pd (II) meso-tetra(pentafluorophenyl) porphyrin; Pt (II) meso-tetra(pentafluorophenyl) porphyrin; Ru (II) tris(4,7-diphenyl-1,10-phenanthroline) (Ru (dpp)₃); Ru (II) tris(1,10-phenanthroline) (Ru(phen)₃), tris(2,2′-bipyridine)rutheniurn (II) chloride hexahydrate (Ru(bpy)₃); erythrosine B; fluorescein; fluorescein isothiocyanate (FITC); eosin; iridium (III) ((N-methyl-benzimidazol-2-yl)-7-(diethylamino)-coumarin)); indium (III) ((benzothiazol-2-yl)-7-(diethylamino)-coumarin))-2-(acetylacetonate); Lumogen dyes; Macroflex fluorescent red; Macrolex fluorescent yellow; Texas Red; rhodamine B; rhodamine 6G; sulfur rhodamine; m-cresol; thymol blue; xylenol blue; cresol red; chlorophenol blue; bromocresol green; bromcresol red; bromothymol blue; Cy2; a Cy3; a Cy5; a Cy5.5; Cy7; 4-nitirophenol; alizarin; phenolphthalein; o-cresolphthalein; chlorophenol red; calmagite; bromo-xylenol; phenol red; neutral red; nitrazine; 3,4,5,6-tetrabromphenolphtalein; congo red; fluorescein; eosin; 2′,7′-dichlorofluorescein; 5 (6)-carboxy-fluorecsein; carboxynaphthofluorescein; 8-hydroxypyrene-1,3,6-trisulfonic acid; semi-naphthorhodafluor; semi-naphthofluorescein; tris(4,7-diphenyl-1,10-phenanthroline) ruthenium (II) dichloride; (4,7-diphenyl-1,10-phenanthroline) ruthenium (II) tetraphenylboron; platinum (II) octaethylporphyin; dialkylcarbocyanine; dioctadecylcycloxacarbocyanine; fluorenylmethyloxycarbonyl chloride; 7-amino-4-methylcourmarin (Amc); green fluorescent protein (GFP); and derivatives or combinations thereof.

In some examples, the detectable moiety can comprise Rhodamine B (Rho), fluorescein isothiocyanate (FITC), 7-amino-4-methylcourmarin (Amc), green fluorescent protein (GFP), naphthofluorescein (NF), or derivatives or combinations thereof.

The detectible moiety can be attached to the cell penetrating peptide moiety at the amino group, the carboxylate group, or the side chain of any of the amino acids of the cell penetrating peptide moiety (e.g., at the amino group, the carboxylate group, or the side chain of any amino acid in the CPP).

### Therapeutic Moiety

The disclosed compounds can also comprise a therapeutic moiety. In some examples, the cargo moiety comprises a therapeutic moiety. The detectable moiety can be linked to a therapeutic moiety or the detectable moiety can also serve as the therapeutic moiety. Therapeutic moiety refers to a group that when administered to a subject will reduce one or more symptoms of a disease or disorder.

The therapeutic moiety can comprise a wide variety of drugs, including antagonists, for example enzyme inhibitors, and agonists, for example a transcription factor which results in an increase in the expression of a desirable gene product (although as will be appreciated by those in the art, antagonistic transcription factors can also be used), are all included. In addition, therapeutic moiety includes those agents capable of direct toxicity and/or capable of inducing toxicity towards healthy and/or unhealthy cells in the body. Also, the therapeutic moiety can be capable of inducing and/or priming the immune system against potential pathogens.

The therapeutic moiety can, for example, comprise an anticancer agent, antiviral agent, antimicrobial agent, anti-inflammatory agent, immunosuppressive agent, anesthetics, or any combination thereof.

The therapeutic moiety can comprise an anticancer agent. Example anticancer agents include 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR. The therapeutic moiety can also comprise a biopharmaceutical such as, for example, an antibody.

In some examples, the therapeutic moiety can comprise an antiviral agent, such as ganciclovir, azidothymidine (AZT), lamivudine (3TC), etc.

In some examples, the therapeutic moiety can comprise an antibacterial agent, such as acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; Lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin; oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillin G benzathine; penicillin G potassium; penicillin G procaine; penicillin G sodium; penicillin V; penicillin V benzathine; penicillin V hydrabamine; penicillin V potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin B sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; or zorbamycin.

In some examples, the therapeutic moiety can comprise an anti-inflammatory agent.

In some examples, the therapeutic moiety can comprise dexamethasone (Dex).

In other examples, the therapeutic moiety comprises a therapeutic protein. For example, some people have defects in certain enzymes (e.g., lysosomal storage disease). It is disclosed herein to deliver such enzymes/proteins to human cells by linking to the enzyme/protein to one of the disclosed cell penetrating peptides. The disclosed cell penetrating peptides have been tested with proteins (e.g., GFP, PTP1B, actin, calmodulin, troponin C) and shown to work.

In some examples, the therapeutic moiety comprises a targeting moiety. The targeting moiety can comprise, for example, a sequence of amino acids that can target one or more enzyme domains. In some examples, the targeting moiety can comprise an inhibitor against an enzyme that can play a role in a disease, such as cancer, cystic fibrosis, diabetes, obesity, or combinations thereof. For example, the targeting moiety can comprise any of the sequences listed in Table 6.

TABLE 6
Example targeting moieties

| Abbreviation * | Sequence |
|---|---|
| PΘGΛYR | Pro-Pip-Gly-F₂Pmp-Tyr-Arg |
| SΘIΛΛR | Ser-Pip-Ile-F₂Pmp-F₂Pmp-Arg |
| IHIΛIR | Ile-His-Ile-F₂Pmp-Ile-Arg |
| AaIΛΘR | Ala-(D-Ala)-Ile-F₂Pmp-Pip-Arg |
| ΣSΘΛvR | Fpa-Ser-Pip-F₂Pmp-(D-Val)-Arg |
| ΘnPΛAR | Pip-(D-Asn)-Pro-F₂Pmp-Ala-Arg |
| TΨAΛGR | Tyr-Phg-Ala-F₂Pmp-Gly-Arg |
| AHIΛaR | Ala-His-Ile-F₂Pmp-(D-Ala)-Arg |
| GnGΛpR | Gly-(D-Asn)-Gly-F₂Pmp-(D-Pro)-Arg |
| fQΘΛIR | (D-Phe)-Gln-Pip-F₂Pmp-Ile-Arg |
| SPGΛHR | Ser-Pro-Gly-F₂Pmp-His-Arg |
| ΘYIΛHR | Pip-Tyr-Ile-F₂Pmp-His-Arg |
| SvPΛHR | Ser-(D-Val)-Pro-F₂Pmp-His-Arg |
| AIPΛnR | Ala-Ile-Pro-F₂Pmp-(D-Asn)-Arg |
| ΣSIΛQF | Fpa-Ser-Ile-F₂Pmp-Gln-Arg |
| AaΨΛfR | Ala-(D-Ala)-Phg-F₂Pmp-(D-Phe)-Arg |
| ntΨΛΨR | (D-Asn)-(D-Thr)-Phg-F₂Pmp-Phg-Arg |
| IPΨΛΩR | Ile-Pro-Phg-F₂Pmp-Nle-Arg |
| QΘΣΛΘR | Gln-Pip-Fpa-F₂Pmp-Pip-Arg |
| nAΣΛGR | (D-Asn)-Ala-Fpa-F₂Pmp-Gly-Arg |
| ntYΛAR | (D-Asn)-(D-Thr)-Tyr-F₂Pmp-Ala-Arg |
| eAΨΛvR | (D-Glu)-Ala-Phg-F₂Pmp-(D-Val)-Arg |
| IvΨΛAR | Ile-(D-Val)-Phg-F₂Pmp-Ala-Arg |
| YtΨΛAR | Tyr-(D-Thr)-Phg-F₂Pmp-Ala-Arg |
| nΘΨΛIR | (D-Asn)-Pip-Phg-F₂Pmp-Ile-Arg |
| ΘnWΛHR | Pip-(D-Asn)-Trp-F₂Pmp-His-Arg |
| YΘvΛIR | Tyr-Pip-(D-Val)-F₂Pmp-Ile-Arg |
| nSAΛGR | (D-Asn)-Ser-(D-Ala)-F₂Pmp-Gly-Arg |
| tnvΛaR | (D-Thr)-(D-Asn)-(D-Val)- |
| | F₂Pmp-(D-Ala)-Arg |
| ntvΛtR | (D-Asn)-(D-Thr)-(D-Val)- |
| | F₂Pmp-(D-Thr)-Arg |
| SItΛYR | Ser-Ile-(D-Thr)-F₂Pmp-Tyr-Arg |
| nΣnΛlR | (D-Asn)-Fpa-(D-Asn)- |
| | F₂Pmp-(D-Leu)-Arg |
| YnnΛΩR | Tyr-(D-Asn)-(D-Asn)-F₂Pmp-Nle-Arg |
| nYnΛGR | (D-Asn)-Tyr-(D-Asn)-F₂Pmp-Gly-Arg |
| AWnΛAR | Ala-Trp-(D-Asn)-F₂Pmp-Ala-Arg |
| vtHΛYR | (D-Val)-(D-Thr)-His-F₂Pmp-Tyr-Arg |
| PΨHΛΘR | Pro-Phg-His-F₂Pmp-Pip-Arg |
| nΨHΛGR | (D-Asn)-Phg-His-F₂Pmp-Gly-Arg |
| PAHΛGR | Pro-Ala-His-F₂Pmp-Gly-Arg |
| AYHΛIR | Ala-Tyr-His-F₂Pmp-Ile-Arg |
| nΘeΛYR | (D-Asn)-Pip-(D-Glu)-F₂Pmp-Tyr-Arg |
| vSSΛtR | (D-Val)-Ser-Ser-F₂Pmp-(D-Thr)-Arg |
| aΞt′ ϑ Φ′YNK | ((D-Ala)-Sar-(D-pThr)- |
| | Pp-Nal-Tyr-Gln)-Lys |
| Tm(aΞt′ϑΦ′RA)Dap | Tm((D-Ala)-Sar-(D-pThr)- |
| | Pp-Nal-Arg-Ala)-Dap |
| Tm(aΞt′ϑΦ′RAa)Dap | Tm((D-Ala)-Sar-(D-pThr)-Pp- |
| | Nal-Arg-Ala-(D-Ala))-Dap |
| Tm(aΞtϑ′RAa)Dap | Tm((D-Ala)-Sar-(D-Thr)-Pp- |
| | Nal-Arg-Ala-(D-Ala))-Dap |
| Tm(aΞtaΦ′RAa)Dap | Tm((D-Ala)-Sar-(D-Thr)-(D-Ala)- |
| | Nal-Arg-Ala-(D-Ala))-Dap |

* Fpa, Σ: L-4-fluorophenylalanine; Pip, Θ: L-homoproline; Nle, Ω: L-norleucine; Phg, Ψ L-phenylglycine; F₂Pmp, Λ: L-4-(phosphonodifluoromethyl)phenylalanine; Dap, L-2,3-diaminopropionic acid; Nal, Φ′: L-β-naphthylalanine; Pp, ϑ: L-pipecolic acid; Sar, Ξ: sarcosine; Tm, trimesic acid.

The targeting moiety and cell penetrating peptide moiety can overlap. That is, the residues that form the cell penetrating peptide moiety can also be part of the sequence that forms the targeting moiety, and vice a versa.

The therapeutic moiety can be attached to the cell penetrating peptide moiety at the amino group, the carboxylate group, or the side chain of any of the amino acids of the cell penetrating peptide moiety (e.g., at the amino group, the carboxylate group, or the side chain or any of amino acid of the CPP). In some examples, the therapeutic moiety can be attached to the detectable moiety.

In some examples, the therapeutic moiety can comprise a targeting moiety that can act as an inhibitor against Ras (e.g., K-Ras), PTP1B, Pin1, Grb2 SH2, CAL PDZ, and the like, or combinations thereof.

Ras is a protein that in humans is encoded by the RAS gene. The normal Ras protein performs an essential function in normal tissue signaling, and the mutation of a Ras gene is implicated in the development of many cancers. Ras can act as a molecular on/off switch, once it is turned on Ras recruits and activates proteins necessary for the propagation of growth factor and other receptors' signal. Mutated forms of Ras have been implicated in various cancers, including lung cancer, colon cancer, pancreatic cancer, and various leukemias.

Protein-tyrosine phosphatase 1B (PTP1B) is a prototypical member of the PTP superfamily and plays numerous roles during eukaryotic cell signaling. PTP1B is a negative regulator of the insulin signaling pathway, and is considered a promising potential therapeutic target, in particular for the treatment of type II diabetes. PIP1B has also been implicated in the development of breast cancer.

Pin1 is an enzyme that binds to a subset of proteins and plays a role as a post phosphorylation control in regulating protein function. Pin1 activity can regulate the outcome of proline-directed kinase signaling and consequently can regulate cell proliferation and cell survival. Deregulation of Pin1 can play a role in various diseases. The up-regulation of Pin1 may be implicated in certain cancers, and the down-regulation of Pin1 may be implicated in Alzheimer's disease. Inhibitors of Pin1 can have therapeutic implications for cancer and immune disorders.

Grb2 is an adaptor protein involved in signal transduction and cell communication. The Grb2 protein contains one SH2 domain, which can bind tyrosine phosphorylated sequences. Grb2 is widely expressed and is essential for multiple cellular functions. Inhibition of Grb2 function can impair developmental processes and can block transformation and proliferation of various cell types.

It was recently reported that the activity of cystic fibrosis membrane conductance regulator (CFTR), a chloride ion channel protein mutated in cystic fibrosis (CF) patients, is negatively regulated by CFTR-associated ligand (CAL) through its PDZ domain (CAL-PDZ) (Wolde, M et al. J. Biol. Chem. 2007, 282, 8099). Inhibition of the CFTR/CAL-PDZ interaction was shown to improve the activity of ΔPhe508-CFTR, the most common form of CFTR mutation (Cheng, S H et al. Cell 1990, 63, 827; Kerem, B S et al. Science 1989, 245, 1073), by reducing its proteasome-mediated degradation (Cushing, P R et al. Angew. Chem. Int. Ed. 2010, 49, 9907). Thus, disclosed herein is a method for treating a subject having cystic fibrosis by administering an effective amount of a compound or composition disclosed herein. The compound or composition administered to the subject can comprise a therapeutic moiety that can comprise a targeting moiety that can act as an inhibitor against CAL PDZ. Also, the dcompositions or compositions disclosed herein can be administered with a molecule that corrects the CFTR function.

In some embodiments, the therapeutic moiety is a nucleic acid. In some embodiments, the nucleic acid is an antisense compound. In some embodiments, the antisense compound is selected from the group consisting of an antisense oligonucleotide, a small interfering RNA (siRNA), microRNA (miRNA), a ribozyme, an immune stimulating nucleic acid, an antagomir, an antimir, a microRNA mimic, a supermir, a Ul adaptor, and an aptamer.

Also disclosed herein are compositions comprising the compounds described herein.

Also disclosed herein are pharmaceutically-acceptable salts and prodrugs of the disclosed compounds. Pharmaceutically-acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically-acceptable base addition salts include sodium, potassium, calcium, ammonium, or magnesium salt. Examples of physiologically-acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The disclosed compounds can be prepared by solid phase peptide synthesis wherein the amino acid α-N-terminus is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for asparticacid and glutamic acid, benzyl and t-butyl and for cysteine, triphenylmethyl (trityl).

In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl) phenoxyacetamidoethyl resin available from Applied Biosystems (Foster City, Calif.). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl) phenoxy-acetamidoethyl resin, the Fmoc group is cleaved with a secondary amine, preferably piperidine, prior to coupling with the α-C-terminal amino acid as described above. One method for coupling to the deprotected 4 (2′,4′-dimethoxyphenyl-Fmoc-aminomethyl) phenoxy-acetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminus in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF. The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleavage reagent comprising thianisole, water, ethanedithiol and trifluoroacetic acid. In cases wherein the α-C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivitized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing.

### Methods of Use

Also provided herein are methods of use of the compounds or compositions described herein. Also provided herein are methods for treating a disease or pathology in a subject in need thereof comprising administering to the subject an effective amount of any of the compounds or compositions described herein.

Also provided herein are methods of treating cancer in a subject. The methods include administering to a subject an effective amount of one or more of the compounds or compositions described herein, or a pharmaceutically acceptable salt thereof. The compounds and compositions described herein or pharmaceutically acceptable salts thereof are useful for treating cancer in humans, e.g., pediatric and geriatric populations, and in animals, e.g., veterinary applications. The disclosed methods can optionally include identifying a patient who is or can be in need of treatment of a cancer. Examples of cancer types treatable by the compounds and compositions described herein include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. Further examples include cancer and/or tumors of the anus, bile duct, bone, bone marrow, bowel (including colon and rectum), eye, gall bladder, kidney, mouth, larynx, esophagus, stomach, testis, cervix, mesothelioma, neuroendocrine, penis, skin, spinal cord, thyroid, vagina, vulva, uterus, liver, muscle, blood cells (including lymphocytes and other immune system cells). Further examples of cancers treatable by the compounds and compositions described herein include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

The methods of treatment or prevention of cancer described herein can further include treatment with one or more additional agents (e.g., an anti-cancer agent or ionizing radiation). The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

For example, the compounds or compositions or pharmaceutically acceptable salts thereof as described herein can be combined into a pharmaceutical composition with an additional anti-cancer agent, such as 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR. The additional anti-cancer agent can also include biopharmaceuticals such as, for example, antibodies.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease.

Also described herein are methods of killing a tumor cell in a subject. The method includes contacting the tumor cell with an effective amount of a compound or composition as described herein, and optionally includes the step of irradiating the tumor cell with an effective amount of ionizing radiation. Additionally, methods of radiotherapy of tumors are provided herein. The methods include contacting the tumor cell with an effective amount of a compound or composition as described herein, and irradiating the tumor with an effective amount of ionizing radiation. As used herein, the term ionizing radiation refers to radiation comprising particles or photons that have sufficient energy or can produce sufficient energy via nuclear interactions to produce ionization. An example of ionizing radiation is x-radiation. An effective amount of ionizing radiation refers to a dose of ionizing radiation that produces an increase in cell damage or death when administered in combination with the compounds described herein. The ionizing radiation can be delivered according to methods as known in the art, including administering radiolabeled antibodies and radioisotopes.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection. Prophylactic administration can be used, for example, in the chemopreventative treatment of subjects presenting precancerous lesions, those diagnosed with early stage malignancies, and for subgroups with susceptibilities (e.g., family, racial, and/or occupational) to particular cancers. Therapeutic treatment involves administering to a subject a therapeutically effective amount of the compounds and compositions or pharmaceutically acceptable salts thereof as described herein after cancer is diagnosed.

In some examples of the methods of treating of treating cancer or a tumor in a subject, the compound or composition administered to the subject can comprise a therapeutic moiety that can comprise a targeting moiety that can act as an inhibitor against Ras (e.g., K-Ras), PTP1B, Pin1, Grb2 SH2, or combinations thereof.

The disclosed subject matter also concerns methods for treating a subject having a metabolic disorder or condition. In one embodiment, an effective amount of one or more compounds or compositions disclosed herein is administered to a subject having a metabolic disorder and who is in need of treatment thereof. In some examples, the metabolic disorder can comprise type II diabetes. In some examples of the methods of treating of treating the metabolic disorder in a subject, the compound or composition administered to the subject can comprise a therapeutic moiety that can comprise a targeting moiety that can act as an inhibitor against PTP1B. In one particular example of this method the subject is obese and the method comprises treating the subject for obesity by administering a composition as disclosed herein.

The disclosed subject matter also concerns methods for treating a subject having an immune disorder or condition. In one embodiment, an effective amount of one or more compounds or compositions disclosed herein is administered to a subject having an immune disorder and who is in need of treatment thereof. In some examples of the methods of treating of treating the immune disorder in a subject, the compound or composition administered to the subject can comprise a therapeutic moiety that can comprise a targeting moiety that can act as an inhibitor against Pin1.

The disclosed subject matter also concerns methods for treating a subject having an inflammatory disorder or condition. In one embodiment, an effective amount of one or more compounds or compositions disclosed herein is administered to a subject having an inflammatory disorder and who is in need of treatment thereof.

The disclosed subject matter also concerns methods for treating a subject having cystic fibrosis. In one embodiment, an effective amount of one or more compounds or compositions disclosed herein is administered to a subject having cystic fibrosis and who is in need of treatment thereof. In some examples of the methods of treating the cystic fibrosis in a subject, the compound or composition administered to the subject can comprise a therapeutic moiety that can comprise a targeting moiety that can act as an inhibitor against CAL PDZ.

In some embodiments, the CPPs disclosed herein can be used for detecting or diagnosing a disease or condition in a subject. For example, a CPP can comprise a targeting moiety and/or a detectible moiety that can interact with a target, e.g., a tumor.

### Compositions, Formulations and Methods of Administration

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 2003/0032594 and 2002/0120100 disclose amino acid sequences that can be coupled to another composition and that allows the composition to be translocated across biological membranes. U.S. Application Publication No. 200/20035243 also describes compositions for transporting biological moieties across cell membranes for intracellular delivery. Compounds can also be incorporated into polymers, examples of which include poly(D-L lactide-co-glycolide) polymer for intracranial tumors; poly[bis (p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The disclosed compositions are bioavailable and can be delivered orally. Oral compositions can be tablets, troches, pills, capsules, and the like, and can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

### EXAMPLES

### Example 1. CPP Synthesis

Purpose. Recently, we discovered that cyclization of CPPs (e.g., Tat and R₉) greatly enhances their cellular uptake efficiency. For example, cyclo(Phe-D-Phe-Nal-Arg-D-Arg-Arg-D-Arg-Gln) (CPP12, where Nal is L-2-naphthylalanine), which is the most active cyclic CPP discovered to date, exhibits a cytosolic delivery efficiency of 120%.

A drawback of CPPs is that their cellular entry kinetics and efficiency are greatly reduced at high concentrations of serum proteins, presumably because binding of the CPPs to serum proteins inhibits their interaction with the cell membrane. For example, the cytosolic entry efficiency of CPP12 into HeLa (human cervical cancer) cells was decreased by 25-fold in the presence of 10% fetal bovine serum (FBS).

Materials. Reagents for peptide synthesis were purchased from Chem-Impex (Wood Dale, IL), NovaBiochem (La Jolla, CA), or Anaspec (San Jose, CA). Rink amide resin (100-200 mesh, 0.43 mmol/g) and fluorescein isothiocyanate (FITC), isomer I, were from Chem-Impex (Wood Dale, IL). 5 (6)-Carboxynaphthofluorescein succinimidyl ester (NF-NHS), was from Setareh Biotech (Eugene, OR). 5 (6)-Carboxytetramethylrhodamine succinimidyl ester (TMR-NHS) and Geneticin™ were from ThermoFisher Scientific (Waltham, MA). Tetrakis(triphenylphosphine)palladium(0) [Pd(PPh₃)₄] was purchased from Sigma-Aldrich (St. Louis, MO) and phenylsilane was from TCI America (Portland, OR). O-Benzotriazole-N,N,N,N-tetramethyluronium hexafluorophosphate (HATU) and (benzotriazol-1-yloxy)-tripyrrolidinophosphonium hexafluorophosphate (PyBOP) were from Matrix Scientific (Columbia, SC). All solvents and other chemical reagents were obtained from Sigma-Aldrich, Fisher Scientific (Pittsburgh, PA), or VWR (West Chester, PA) and were used without further purification. Cell culture media, fetal bovine serum (FBS), penicillin-streptomycin, 0.25% trypsin-EDTA, DPBS, 100× non-essential amino acids, and sodium pyruvate solution were from Sigma-Aldrich. HeLa cells were obtained from ATCC (Manassas, VA). ARE reporter (Luc)-HepG2 cell line and One-Step™ luciferase assay system were purchased from BPS Bioscience (San Diego, CA). The cell proliferation kit [3-(4,5-dimethylthiazol-2-yl)-2,5-diphenyltetrazolium bromide (MTT)] was purchased from Roche (Indianapolis, IN).

General Methods-Peptide Synthesis. Peptides were synthesized manually on Rink amide resin (0.43 mmol/g) using standard Fmoc chemistry. The typical coupling reaction contained 5 eq. of Fmoc-amino acid, 5 eq. of HATU, and 10 eq. of diisopropylethylamine (DIPEA) and was allowed to proceed with mixing for 30 min at room temperature (RT). For cyclic CPPs, after the addition of the last (N-terminal) residue, the allyl group on the C-terminal Glu residue was removed by treatment with 0.3 eq. Pd(PPh₃)₄ and 10 eq. phenylsilane in anhydrous DCM in the dark (3×15 min). The resin was washed twice with sodium dimethyldithiocarbamate dihydrate (SDDCM, 0.5 M in DMF) and the N-terminal Fmoc group was removed by treatment with 20% piperidine in DMF. The resin was extensively washed with DMF and DCM and incubated in 1 M 1-hydroxybenzotriazole (HOBt) in DMF for 20 min. The peptide was cyclized by using 10 eq. PyBOP, 10 eq. HOBT, 20 eq. DIPEA in DMF for 1 h at RT. For CPP-P1 conjugates, Fmoc-Lys (Mtt)-OH was first added at the C-terminus, to serve as a linker for conjugation of cyclic CPP and cyclic peptide P1. The linear CPP sequence was synthesized using standard Fmoc chemistry. The CPP sequence was cyclized as described above. The 4-methyltrityl (Mtt) group on the C-terminal Lys was removed by treatment with 2% trifluoroacetic acid (TFA and 1% triisopropylsilane in DCM (6×5 min). Fmoc-8-amino-3,6-dioxaoctanoic acid (miniPEG) was coupled to the Lys side chain, followed by the synthesis of the linear sequence of P1. The allyl protecting group on the L-Glu and the Fmoc group of the N-terminal Gly residue were removed as described above. The linear sequence of P1 was cyclized on resin as described above. Cleavage and deprotection of the completed peptide sequences were performed on resin using 92.5/2.5/2.5/2.5 (v/v) TFA/triisopropylsilane/1,3-dimethoxybenzene/water for 3 h at RT. The peptides were triturated with cold diethyl ether (3 times) and purified by reversed-phase HPLC on a semi-preparative Waters XBridge C18 column. The purity of the peptides (≥95%) was assessed by reversed-phase HPLC equipped with an analytical Waters XBridge C18 column. Peptide authenticity was confirmed by MALDI FT-ICR mass spectrometry at Campus Chemical Instrumentation Center of The Ohio State University. The structures of the peptides studied are shown below.

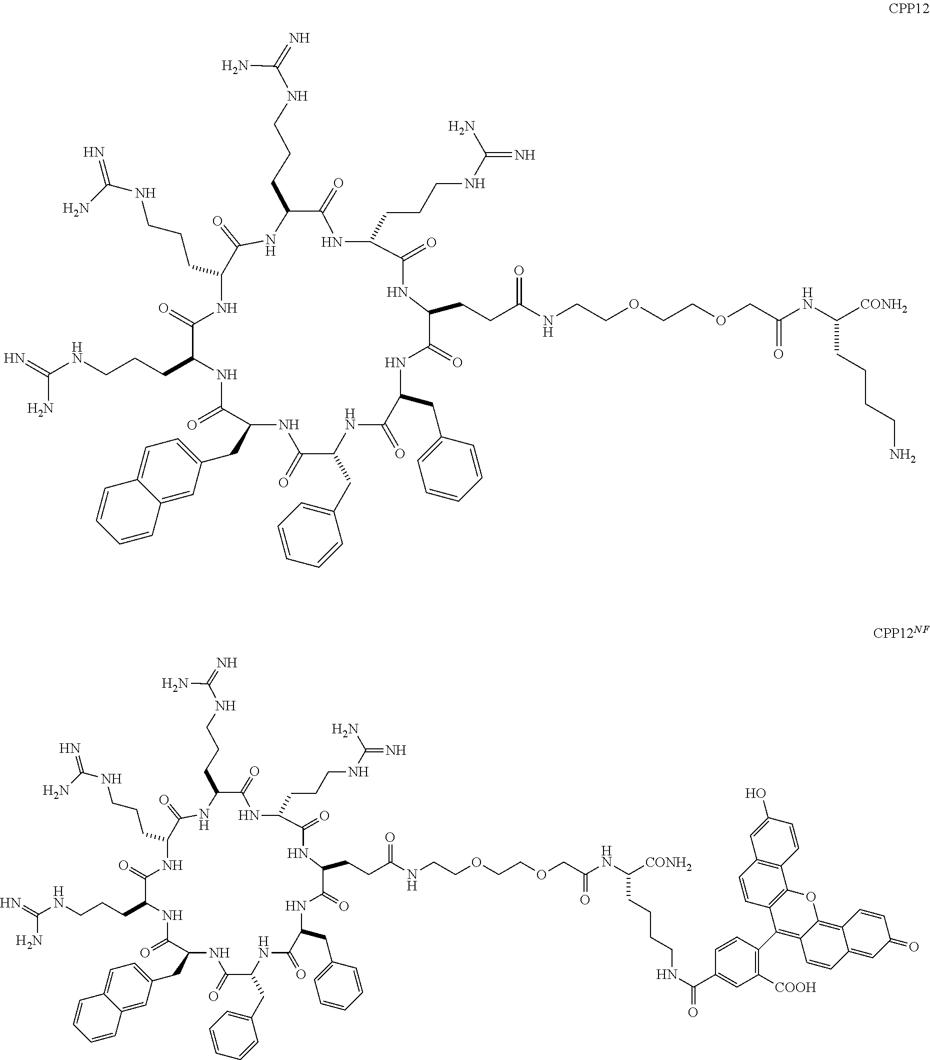

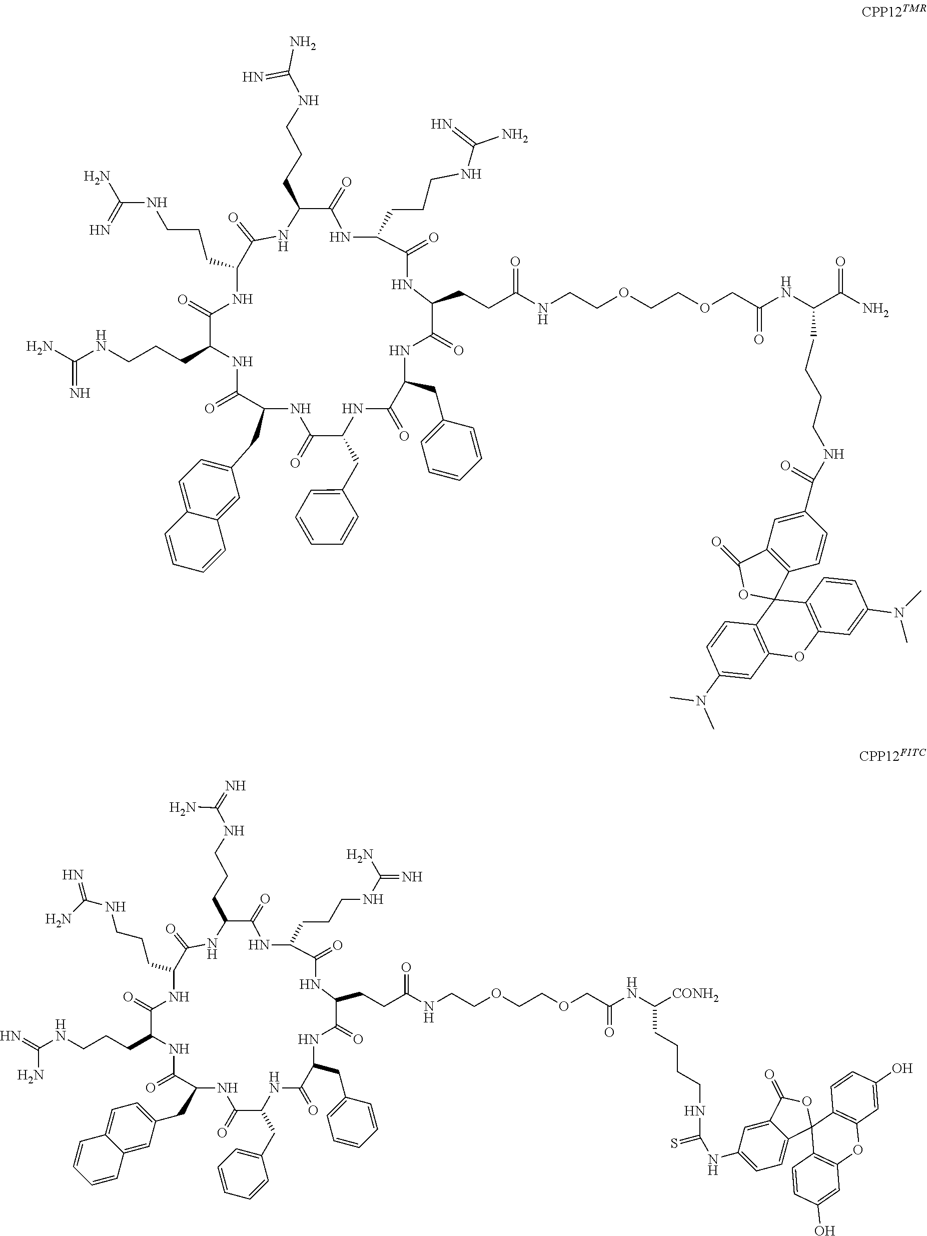

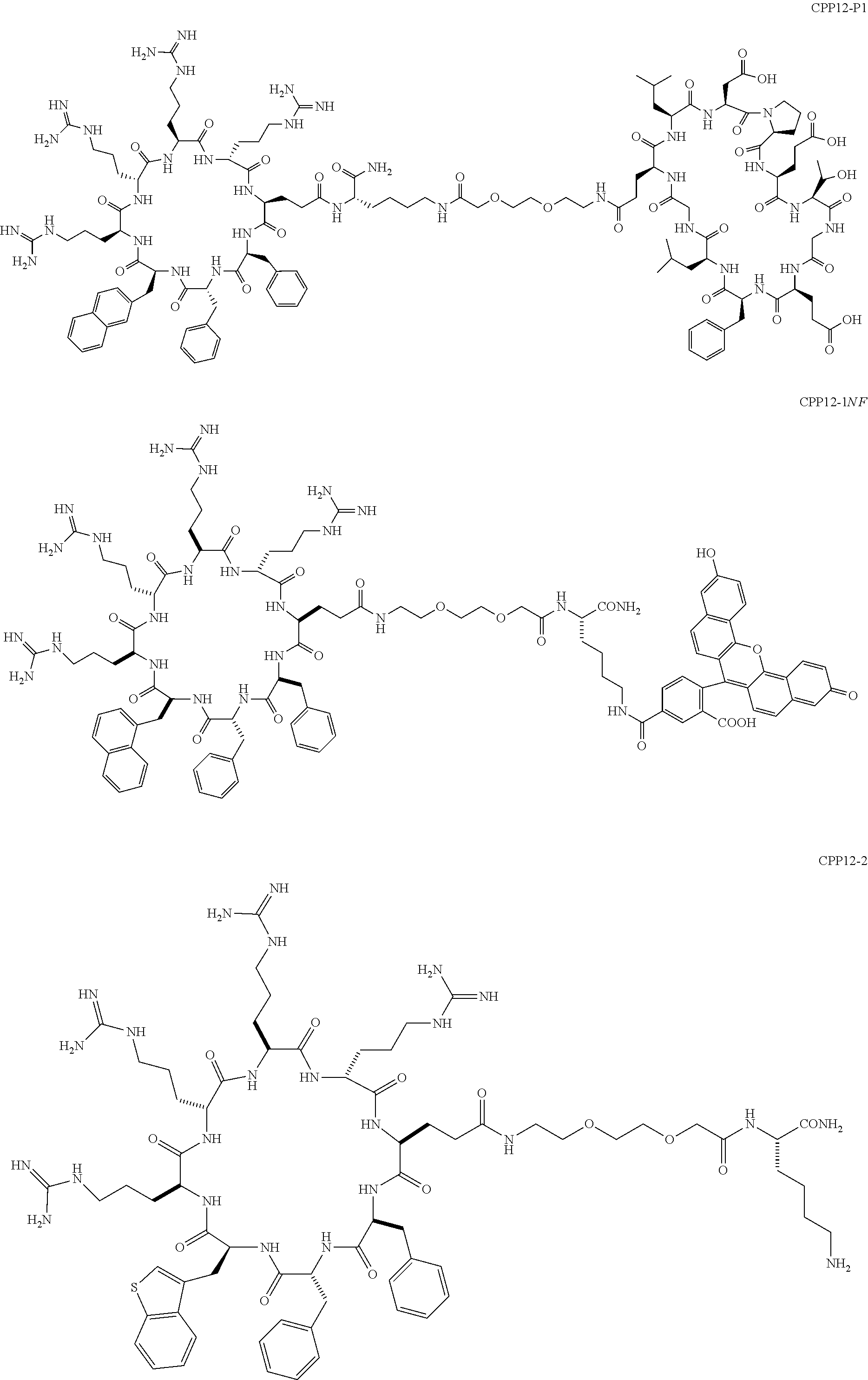

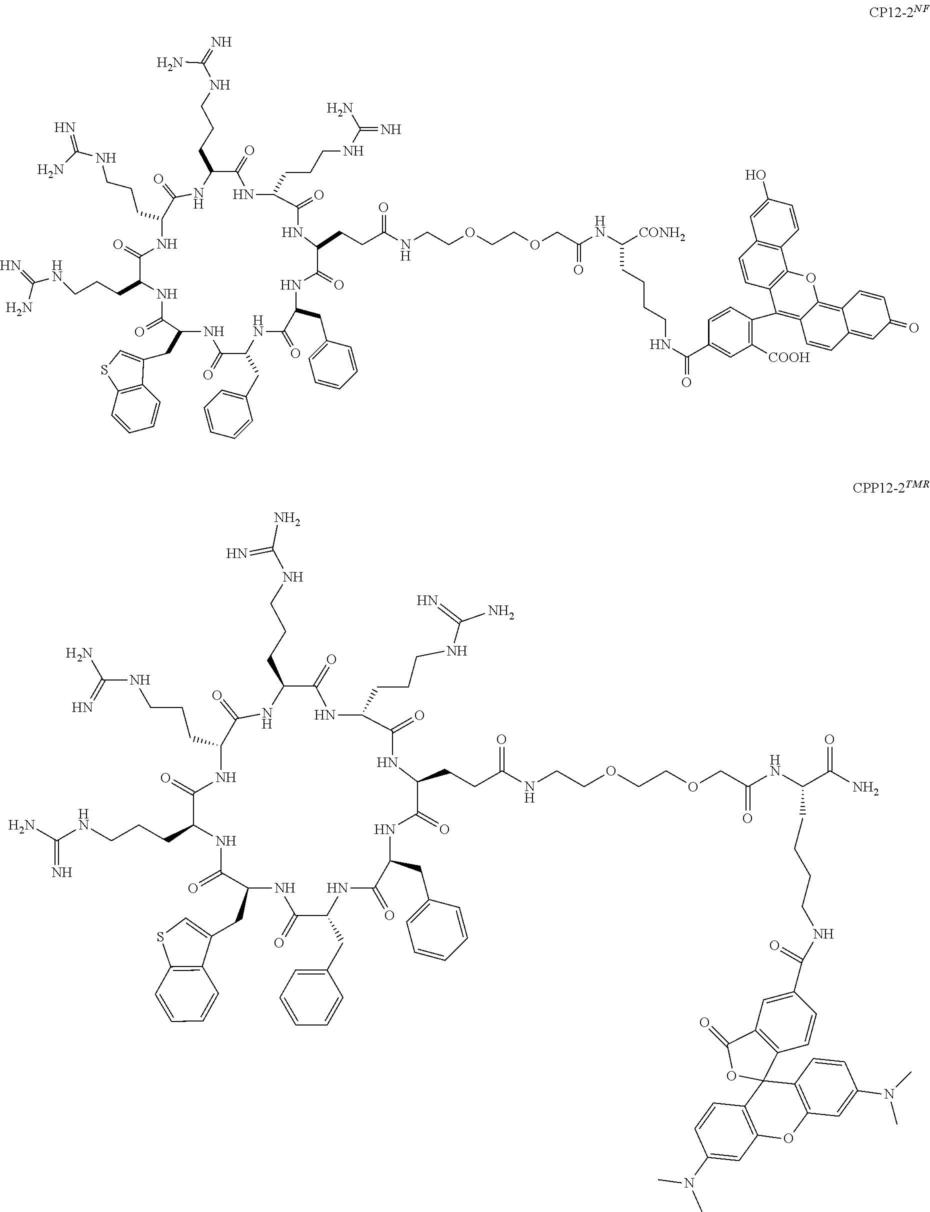

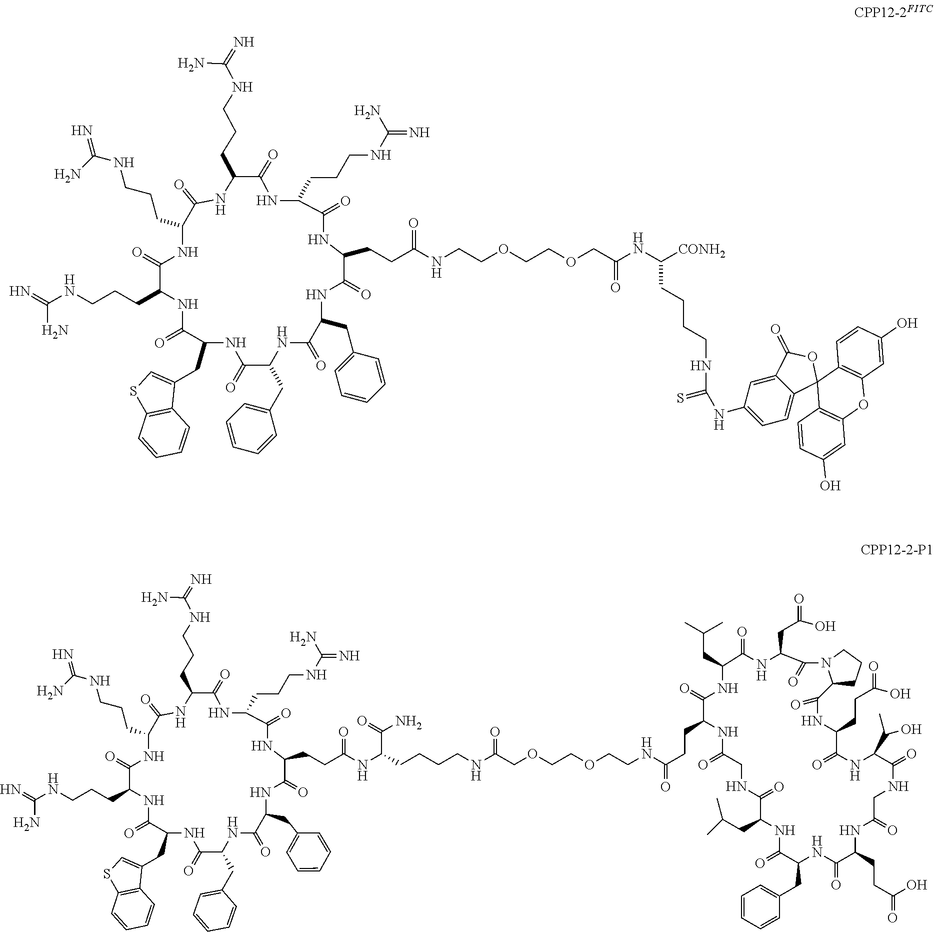

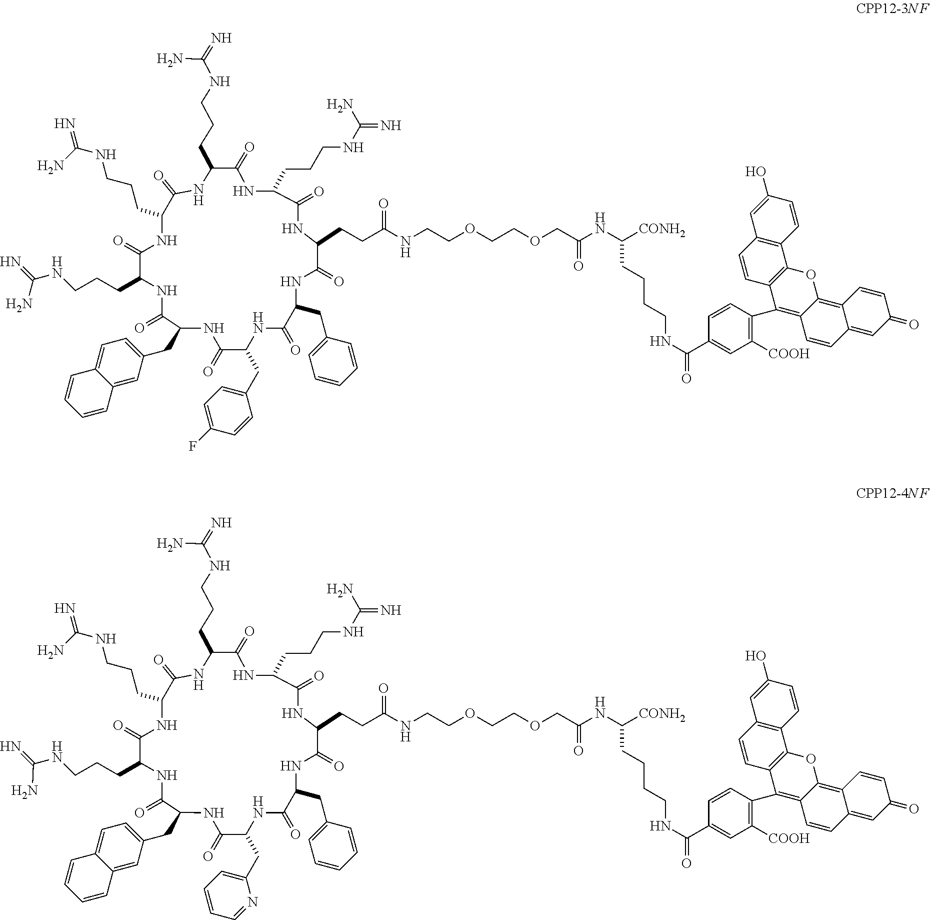

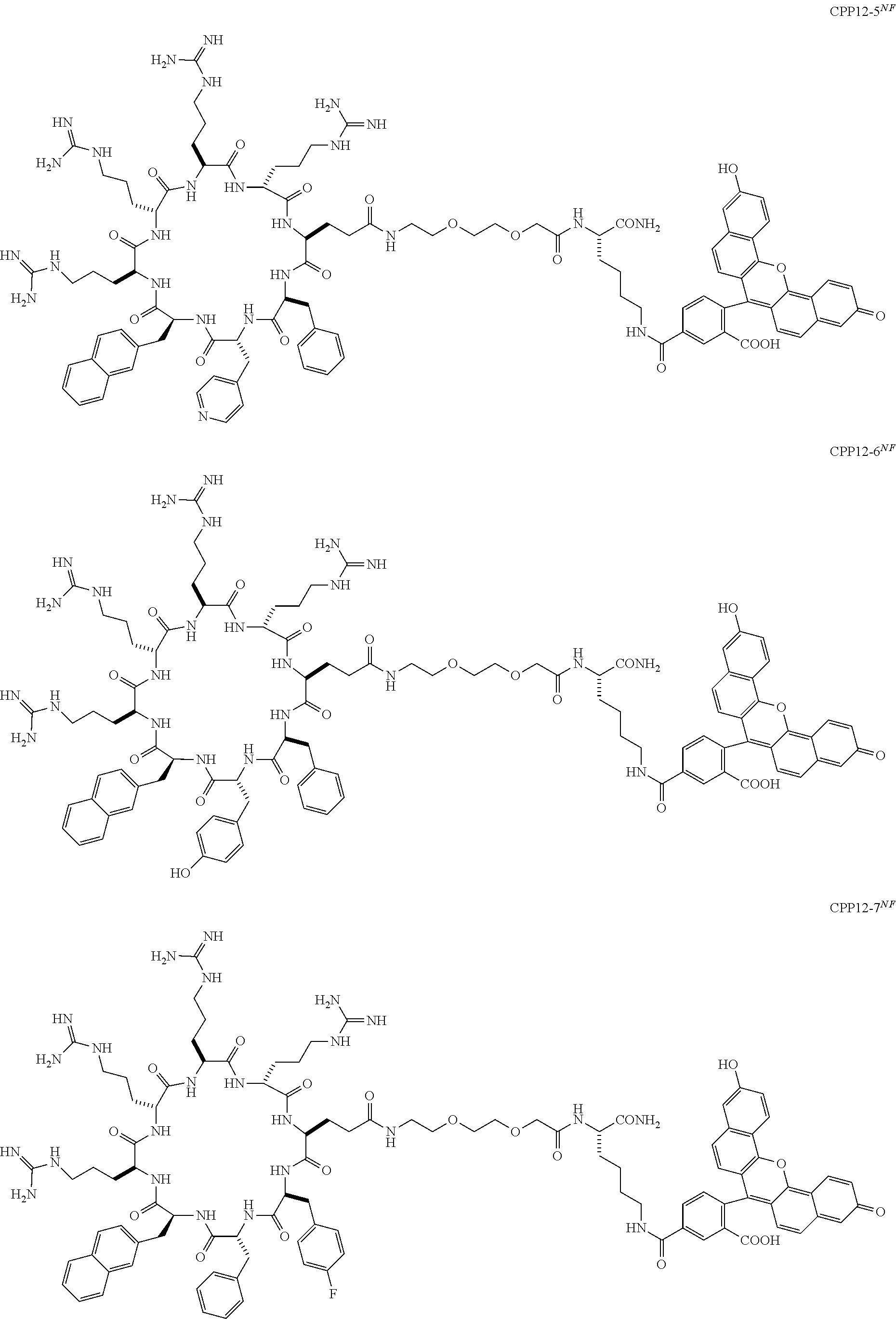

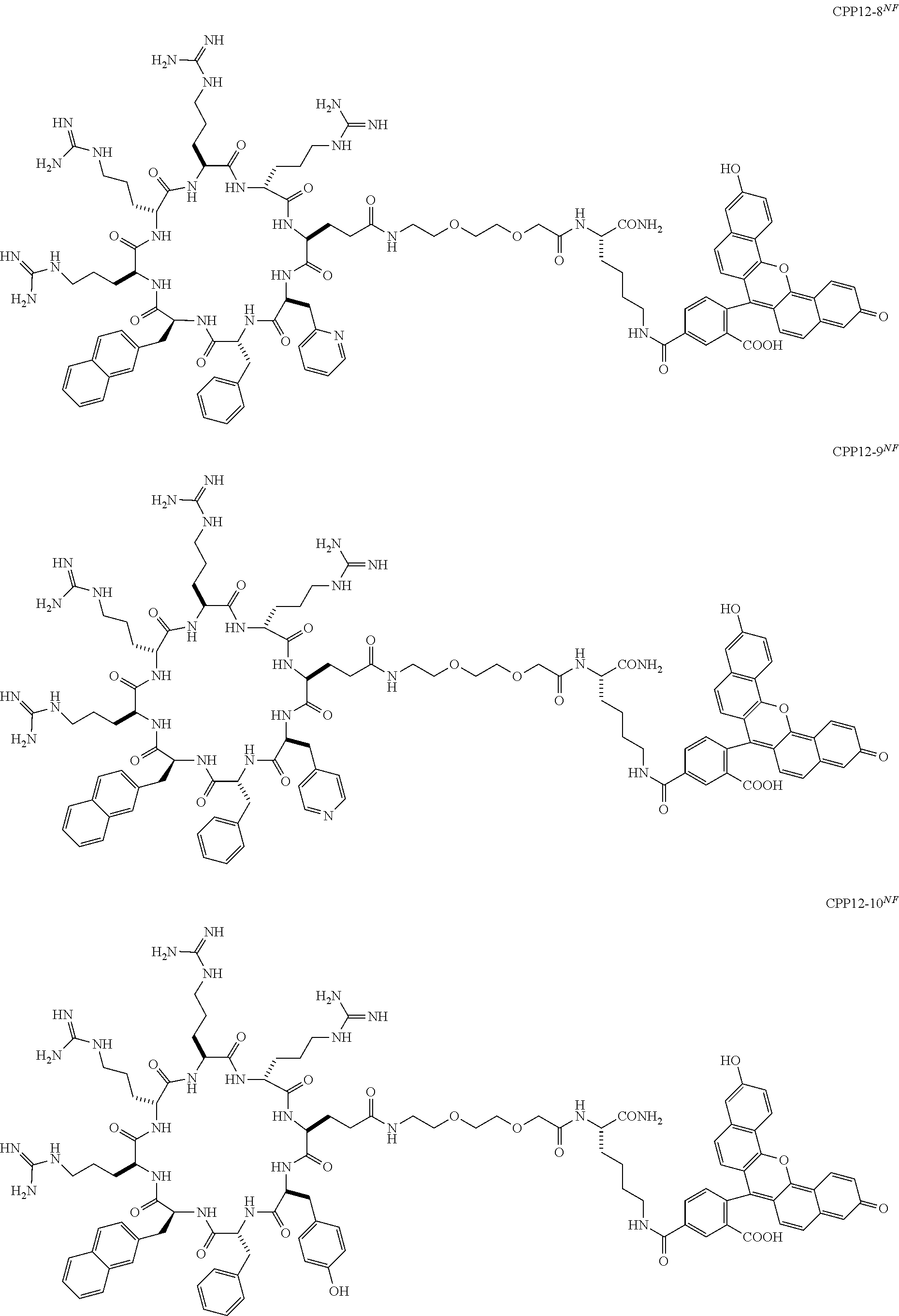

Table A shows the purity and mass spectrometry data of CPP12 and analogues thereof.

TABLE A
Purity and MS data of CPP12 and Analogs CPP12-1 to CPP12-10

| | Molecular | | Calculated | Observed |
|---|---|---|---|---|
| | Formula | Purity | Mass | Mass |
| Compound ID | [M + H]⁺ | (%) | [M + H]⁺ | [M + H]⁺ |
| CPP12 | C₇₂H₁₀₉N₂₄O₁₃⁺ | 98.6 | 1517.8600 | 1517.8614 |
| CPP12^{NF} | C₁₀₁H₁₂₃N₂₄O₁₉⁺ | 99.7 | 1975.9391 | 1975.9277 |
| CPP12^{TMR} | C₉₇H₁₂₉N₂₆O₁₇⁺ | 98.5 | 1930.0024 | 1930.0033 |
| CPP12^{FITC} | C₉₃H₁₂₀N₂₅O₁₈S⁺ | 100 | 1906.8958 | 1906.8893 |
| CPP12-P1 | C₁₂₅H₁₈₄N₃₅O₃₂⁺ | 96.6 | 2687.3841 | 2687.3567 |
| CPP12-1^{NF} | C₁₀₁H₁₂₃N₂₄O₁₉⁺ | 100 | 1975.9391 | 1975.9334 |
| CPP12-2 | C₇₀H₁₀₇N₂₄O₁₃S⁺ | 95.8 | 1523.8165 | 1523.8121 |
| CPP12-2^{NF} | C₉₉H₁₂₁N₂₄O₁₃S⁺ | 97.4 | 1981.8955 | 1981.8901 |
| CPP12-2^{TMR} | C₉₅H₁₂₇N₂₆O₁₇S⁺ | 96.1 | 1935.9588 | 1935.9559 |
| CPP12-2^{FITC} | C₉₁H₁₁₈N₂₅O₁₈S2⁺ | 98.4 | 1912.8523 | 1912.8412 |
| CPP12-2-P1 | C₁₂₃H₁₈₂N₃₅O₃₂S⁺ | 96.9 | 2693.3405 | 2693.3141 |
| CPP12-3^{NF} | C₁₀₁H₁₂₂FN₂₄O₁₉⁺ | 100 | 1993,9297 | 1993.9236 |
| CPP12-4^{NF} | C₁₀₀H₁₂₂N₂₅O₁₉⁺ | 97.7 | 1976.9343 | 1976.9270 |
| CPP12-5^{NF} | C₁₀₀H₁₂₂N₂₅O₁₉⁺ | 99.4 | 1976.9343 | 1976.9250 |
| CPP12-6^{NF} | C₁₀₁H₁₂₃N₂₄O₂₀⁺ | 95.3 | 1991.9340 | 1991.9216 |
| CPP12-7^{NF} | C₁₀₁H₁₂₂FN₂₄O₁₉⁺ | 95.3 | 1993.9297 | 1993.9198 |
| CPP12-8^{NF} | C₁₀₀H₁₂₂N₂₅O₁₉⁺ | 99.1 | 1976.9343 | 1976.9240 |
| CPP12-9^{NF} | C₁₀₀H₁₂₂N₂₅O₁₉⁺ | 98.0 | 1976.9343 | 1976.9291 |
| CPP12-10^{NF} | C₁₀₁H₁₂₃N₂₄O₂₀⁺ | 96.5 | 1991.9340 | 1991.9270 |

General Methods-Fluorescent Labeling of Peptides. Peptide labeling with NF-NHS, TMR-NHS, or FITC was carried out in the solution phase. Lyophilized CPP (1 mg) was dissolved in 25 μL of DMF and the pH was adjusted to 8.0 (for NF-NHS or TMR-NHS labelling) or 8.5 (for FITC labelling) by the addition of a 0.1 M sodium bicarbonate solution. Three eq. of NF-NHS, TMR-NHS, or FITC was dissolved in 25 μL of DMF and added to the peptide solution. The mixture was incubated at RT for 2 h on a shaker. The labeled peptides were purified by HPLC and analyzed by MALDI FT-ICR mass spectrometry as previously described.

General Methods—Cell Culture. HeLa cells were cultured in Dulbecco's modified eagle's medium (DMEM) supplemented with 10% FBS and 1% penicillin-streptomycin sulfate. ARE reporter (Luc)-HepG2 cells were cultured in DMEM supplemented with 10% FBS, 1% penicillin-streptomycin sulfate, 1% non-essential amino acids, 1 mM sodium pyruvate, and 600 μg/mL Geneticin. Cells were cultured in a humidified incubator at 37° C. in the presence of 5% CO₂.

General Methods—Flow Cytometry. HeLa cells were seeded in a 12-well plate at a density 1.5×10⁵ cells per well and cultured overnight. Next day, fluorescently labeled peptide was added in DMEM supplemented with 1% or 10% FBS and 1% penicillin-streptomycin sulfate and incubated at 37° C. for 2 h. After incubation, the cells were washed with cold DPBS twice, detached from the plate with 0.25% trypsin, diluted into cold DPBS and pelleted at 300 g for 5 min at 4° C. The supernatant was discarded and the cells were washed twice with cold DPBS and resuspended in 200 μL of cold DPBS. The samples were analyzed on a BD FACS LSR II flow cytometer. For the FITC-labelled peptides, a 488-nm laser was used for excitation and the fluorescence was analyzed in the FITC channel. For the TMR-labelled peptides, a 561-nm laser was used for excitation and the fluorescence was analyzed in the PE channel. For NF-labelled peptides, a 633-nm laser was used for excitation and the fluorescence emission was analyzed in the APC channel.

General Methods—Endosomal Escape Efficiency. The apparent endosomal escape efficiency of CPP12-2, relative to that of CPP12, was calculated by using equation:

$$γ = \frac{{MFI}_{12 - 2}^{NF} / {MFI}_{12 - 2}^{TMR}}{{MFI}_{12}^{NF} / {MFI}_{12}^{TMR}} \times 100 \%$$

where MFI₁₂^{NF} and MFI₁₂₋₂^{NF} represent the mean fluorescence intensity (MFI) values of HeLa cells treated with NF-labelled CPP12 and CPP12-2, respectively, while MFI₁₂^{TMR} and MFI₁₂₋₂^{TMR} are the corresponding values for cells treated with TMR-labelled CPPs.

General Methods—Confocal Microscopy. HeLa cells were seeded in a 35-mm glass-bottomed microwell dish with 4 compartments (Greiner) at a density of 5×10⁴ cells/mL (300 μL in each compartment) and cultured overnight. Next day, the cells were gently washed with DPBS twice, and treated for 2 h with FITC- or TMR-labelled peptides (5 μM, 1 μM or 0.2 μM as indicated) in phenol-red free DMEM containing 1% and 1% penicillin-streptomycin sulfate. After removal of the medium, the cells were gently washed with DPBS twice and imaged on a Nikon A1R live-cell confocal laser scanning (ECLIPSE Ti-E automated, inverted) microscope equipped with a 100× oil objective (1.45 N.A.) and a heated (37° C.) chamber supplied with 5% CO₂. For the red channel (TMR), the laser line with λ_{Ex} 561 nm was set at 0.6% laser power (for 5 μM peptide treatment), 1.6% laser power (for 1 μM peptide treatment) or 10.8% laser power (for 0.2 μM peptide treatment). For the green channel (FITC), the laser line with λ_{Ex} 487 nm was set at 0.6% laser power. The data were analyzed using NIS-Elements AR.

General Methods—MTT Cell Viability Assay. HeLa cells were seeded in a transparent 96-well plate at a density of 5×10³ cells/well (100 μL in each well) in DMEM containing 10% FBS and 1% penicillin-streptomycin sulfate and cultured overnight. Next day, cells were treated with varying concentrations of peptide (0-50 μM) and incubated at 37° C. with 5% CO₂ for 72 h. Ten μL of MTT solution (0.5 mg/mL) was added to each well and incubated for 4 h, followed by addition of 100 μL of the SDS-HCl solubilizing buffer with thorough mixing and overnight incubation at 37° C. The absorbance of the formazan product was measured at 565 nm on a Tecan M1000 plate reader.

General Methods—Fluorescence Polarization (FP) Analysis of CPP Binding to FBS. Fluorescein-labeled peptide (100 nM) was incubated with serial dilutions of FBS (0-100 μM) in DPBS for 1 h. The solutions were transferred to a 384-black microplate (Greiner), and FP values were measured on a Tecan Infinite M1000 Pro plate reader, with excitation and emission wavelengths at 470 and 535 nm, respectively, for FITC, 530 and 580 nm for TMR, and 595 and 660 nm for NF. The titration curves were fitted using GraphPad Prism to the following equation:

$$FP = \frac{( {A}_{min} + ( {A}_{max} \times \frac{{Q}_{b}}{{Q}_{f}} - {A}_{min} ) ( \frac{( L + x + {K}_{d} ) - \sqrt{( {( L + x + {K}_{d} )}^{2} - 4 Lx )}}{2 L} ) )}{( 1 + ( \frac{{Q}_{b}}{{Q}_{f}} - 1 ) ( \frac{( L + x + {K}_{d} ) - \sqrt{( {( L + x + {K}_{d} )}^{2} - 4 Lx )}}{2 L} ) )}$$

where FP is the measured polarization, Aₘᵢₙ is the minimum FP value, Aₘₐₓ is the maximum FP value, Q_{b} is the quantum yield of the bound fluorophore, Q_{f} is the quantum yield of the free fluorophore, L is the ligand concentration, K_{D} is the dissociation constant, and x is the protein concentration. Data presented are the mean±SD of three independent experiments.

General Methods—FP-based Competition Assay. The binding affinity of P1 and CPP-P1 conjugates to Keap1 was determined by FP-based competition assay. Fluorescein-labelled peptide 2 (20 nM) was incubated for 1 h with 40 nM Keap1 protein in 20 mM HEPES, pH 7.5, 150 mM NaCl, 5 mM dithiothreitol, and 0.01% Triton-X. Serial dilutions of competing peptide were prepared in the same buffer. Aliquots of the equilibrated peptide probe-protein mixture were mixed with each peptide dilution and incubated for 1 h. The samples were transferred to a 384-black microplate (Greiner) and FP values were measured on a Tecan Infinite M1000 Pro plate reader. The data were analyzed by GraphPad Prism with log [inhibitor] vs. response (four parameters).

General Methods—Luciferase Reporter Assay. ARE reporter-HepG2 cells (1000 cells per well) were seeded in 100 μL of assay medium (MEM, 0% FBS, and 1% penicillin-streptomycin sulfate) in an opaque 96-well plate and cultured overnight. Cells were treated with varying concentrations of P1 and CPP-P1 conjugates (0-5 μM) for 18 h at 37° C. in the presence of 5% CO₂. After incubation, 100 μL of One-Step luciferase assay reagent was added to each well and after 15 min of shaking, the luminescence was measured using a Tecan Infinite M1000 Pro microplate reader. P values (<0.05) were estimated using Student's t test.

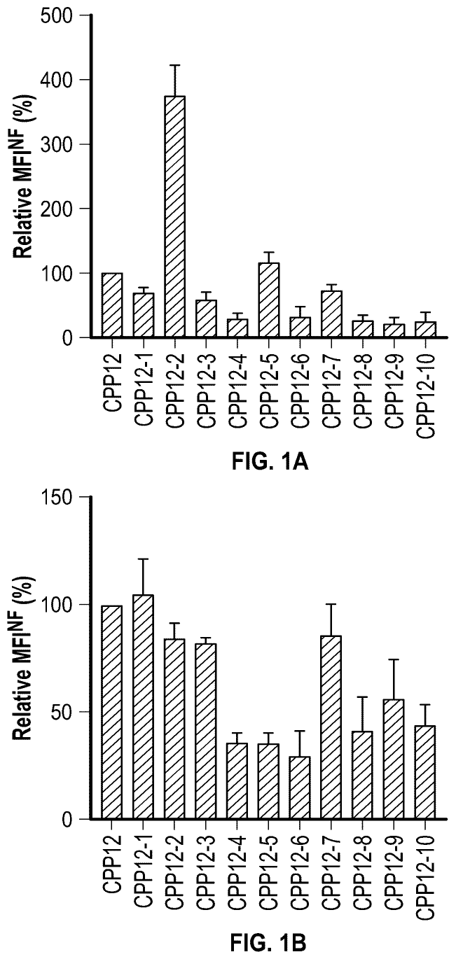

Results—Discovery of CPP12 Analogs of Improved Cytosolic Entry Efficiency. To reduce the protein binding of CPP12 and potentially improve its cellular entry efficiency at high serum concentrations, we synthesized a series of CPP12 analogs by replacing its hydrophobic residues with other amino acids of similar or lower hydrophobicity (peptides CPP12-1 to CPP12-10, Table A). Each peptide was labeled with naphthofluorescein (NF) at the Gln side chain through a long, flexible linker, miniPEG-Lys. NF is a pH-sensitive dye with a pKa of ˜7.8, which is protonated and non-fluorescent inside the acidic environments of endosomes and lysosomes (pH 4.5-6.5) but becomes fluorescent upon entering the cytosol and nucleus (pH 7.4). HeLa cells were incubated with 5 μM NF-labeled peptides for 2 h in the presence of 10% FBS and washed extensively to remove any extracellular peptide. The cells were analyzed by flow cytometry and the cytosolic entry efficiencies of the CPP12 analogs (relative to that of CPP12, which is defined as 100%) were calculated from the MFI_{CPP}^{NF}/MFI_{CPP12}^{NF} ratios. Replacement of 2-Nal with L-1-naphthylalanine (1-Nal; Table 1, CPP12-1) decreased its CPP activity by 1.4-fold, whereas substitution of L-3-benzothienylalanine (Bta; Table A, CPP12-2) resulted in a 3.8-fold improvement in the cytosolic entry efficiency over CPP12. Substitution of D-4-fluorophenylalanine (D-Fpa) for D-Phe (CPP12-3) or L-4-fluorophenylalanine (Fpa) for Phe (Table B and FIG. 1A and FIG. 1B), or replacement of Phe or D-Phe with more hydrophilic residues [L-/D-Tyr or L-/D-2- or 4-pyridylalanine (Pya)] decreased the CPP activity by more than 3-fold (CPP12-4, -6, -8, -9, -10), except for CPP12-5, which showed slightly improved cytosolic entry efficiency.

TABLE B
Structures and Cytosolic Entry Efficiencies of CPP12 Analogs

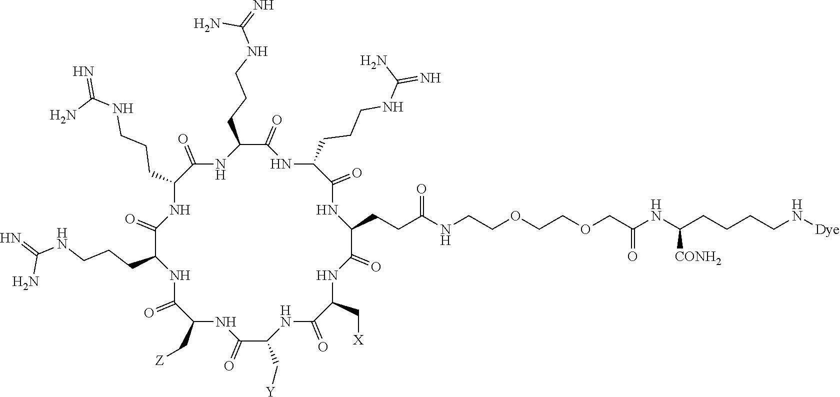

| | | | | Cytosolic Entry | Cytosolic Entry |
|---|---|---|---|---|---|
| Peptide | Peptide Sequenceª | | | Efficiency in 10% | Efficiency in 1% |
| No. | X = | Y = | Z = | FBS (MFI^{NF}, %)^{b} | FBS (MFI^{NF}, %)^{b} |
| CPP12 | Phe | D-Phe | 2-Nal | 100 | 100 |
| CPP12-1 | Phe | D-Phe | 1-Nal | 69 ± 9 | 105 ± 16 |
| CPP12-2 | Phe | D-Phe | Bta | 375 ± 48 | 85 ± 7 |
| CPP12-3 | Phe | D-Fpa | 2-Nal | 59 ± 12 | 83 ± 2 |
| CPP12-4 | Phe | D-2-Pya | 2-Nal | 29 ± 9 | 35 ± 5 |
| CPP12-5 | Phe | D-4-Pya | 2-Nal | 115 ± 18 | 36 ± 5 |
| CPP12-6 | Phe | D-Tyr | 2-Nal | 32 ± 15 | 30 ± 12 |
| CPP12-7 | Fpa | D-Phe | 2-Nal | 74 ± 9 | 86 ± 15 |
| CPP12-8 | 2-Pya | D-Phe | 2-Nal | 27 ± 8 | 41 ± 16 |
| CPP12-9 | 4-Pya | D-Phe | 2-Nal | 22 ± 9 | 57 ± 18 |
| CPP12-10 | Tyr | D-Phe | 2-Nal | 25 ± 15 | 44 ± 10 |

^{a}Bta, L-3-benzothienylalanine; Fpa, L-4-fluorophenylalanine; D-Fpa, D-4-fluorophenylalanine; 1-Nal, L-1-naphthylalanine; 2-Nal, L-2-naphthylalanine; 2-Pya, L-2-pyridylalanine; D-2-Pya, D-2-pyridylalanine; 4-Pya, L-4-pyridylalanine; D-4-Pya, D-4-pyridylalanine.
^{b}All values are relative to that of CPP12 (100%) and represent the mean ± SD of three independent experiments.

We next assessed the “intrinsic” cytosolic entry efficiencies of the CPP12 analogs by repeating the flow cytometry experiments in the presence of reduced serum concentration (1% FBS). Replacement of 2-Nal with 1-Nal (CPP12-1) did not significantly change the intrinsic cytosolic entry efficiency, while substitution of Bta for 2-Nal (CPP12-2) or D-/L-Fpa for D-/L-Phe (CPP12-3 and CPP12-7) slightly reduced the CPP activity (by 14% to 17%) (Table B). Again, substitution of more hydrophilic residues for Phe or D-Phe substantially reduced the intrinsic CPP activity of the CPP12 (by 2- to 3-fold). Interestingly, contrary to our original hypothesis, most of the analogs were “more sensitive” to serum proteins (performed worse at higher FBS concentration) than CPP12, whereas CPP12-2 and CPP12-5 were notable exceptions. We selected CPP12-2 for further evaluation, as it showed substantially improved performance over CPP12 at 10% FBS.

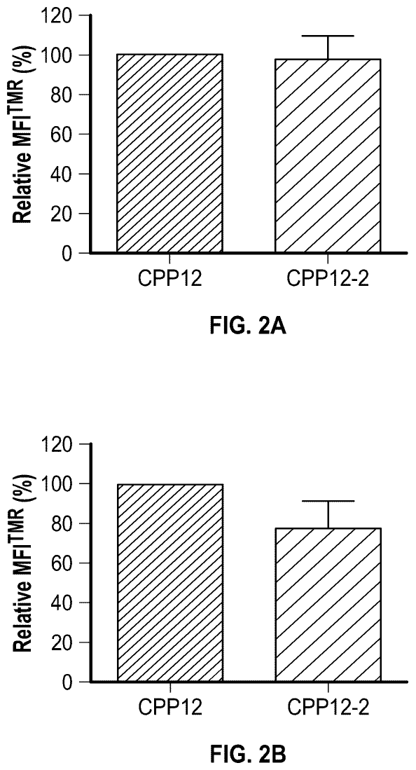

Results—CPP12-2 Has Improved Endosomal Escape Efficiency. The improved cytosolic entry efficiency of CPP12-2 at high serum concentrations can potentially be caused by higher endocytic uptake, more efficient endosomal escape, or both. To differentiate these possibilities, we labeled CPP12 and CPP12-2 with a pH-insensitive dye, tetramethylrhodamine (TMR), treated HeLa cells with 5 μM TMR-labeled peptide for 2 h, and quantitated the total cellular uptake of CPP12^{TMR} and CPP12-2^{TMR} by flow cytometry. CPP12-2 showed similar total cellular uptake to CPP12 in the presence of 10% FBS (98% relative to that of CPP12) but slightly lower uptake in the presence of 1% FBS (79%) (Table C and FIG. 2A and FIG. 2B). The relative endosomal escape efficiency of CPP12-2 was then calculated from the MFI^{NF}/MFI^{TMR} ratio. CPP12-2 exhibited relative endosomal escape efficiencies of 108% and 383% in the presence of 1% and 10% FBS, respectively [relative to that of CPP12 (100%)]. These results reveal that the higher cytosolic entry efficiency of CPP12-2 than CPP12 at high serum concentrations is primarily the result of improved endosomal escape efficiency. However, its different endosomal escape efficiencies in the presence of 1% vs 10% FBS was intriguing.

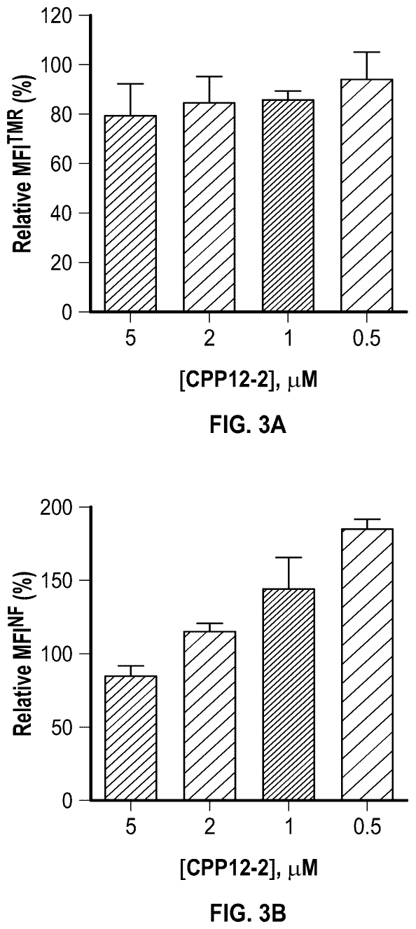

Without being bound by theory, the above observation can potentially be explained by the improved endosomal escape efficiency of CPP12-2 (relative to that of CPP12) at lower peptide concentrations, as high serum concentrations result in binding/sequestration of the CPP, reducing the free CPP concentration. To test this notion, we treated HeLa cells with different concentrations of CPP12-2^{TMR} or CPP12-2^{NF} (5.0, 2.0, 1.0, and 0.5 μM) in the presence of 1% FBS and quantitated the total cellular uptake and cytosolic entry efficiencies as a function of CPP concentration. Although both the total cellular uptake and cytosolic entry efficiencies of CPP12-2 (relative to CPP12) decreased with the CPP concentration, the magnitude of reduction was greater for cytosolic entry (from 186% at 0.5 μM to 85% at 5.0 μM) than total cellular uptake (from 94% to 79%) (Table C and FIG. 3A and FIG. 3B). Finally, we calculated the apparent endosomal escape efficiencies of CPP12-2 (relative to that of CPP12) at different CPP concentrations. In agreement with our hypothesis, CPP12-2 showed the highest relative endosomal escape efficiency (198% of that of CPP12) at 0.5 μM CPP12-2, which progressively decreased with the CPP concentration (to 108% at 5 μM). Thus, CPP12-2 has higher endosomal escape efficiency than CPP12 under all CPP concentrations tested, but especially at lower CPP concentrations.

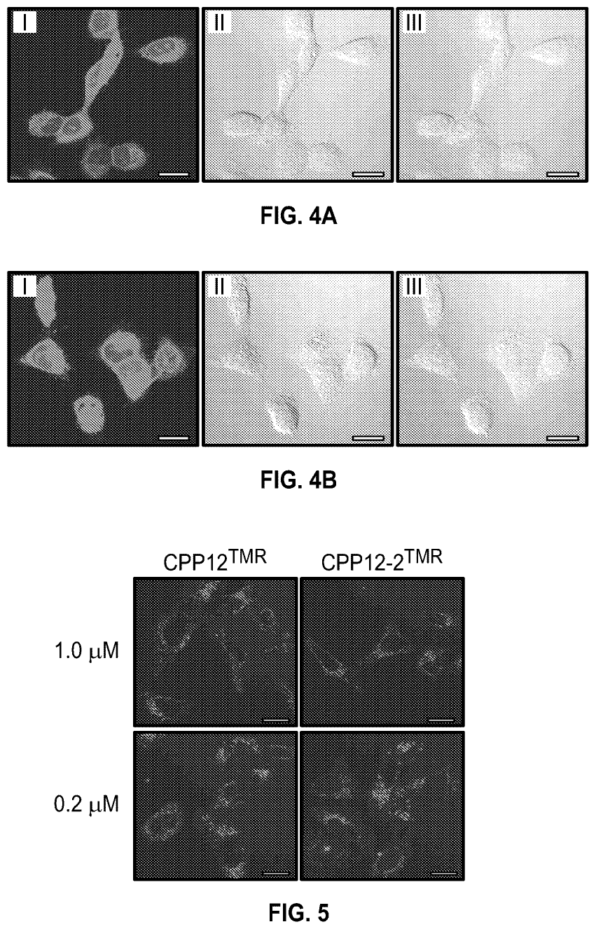

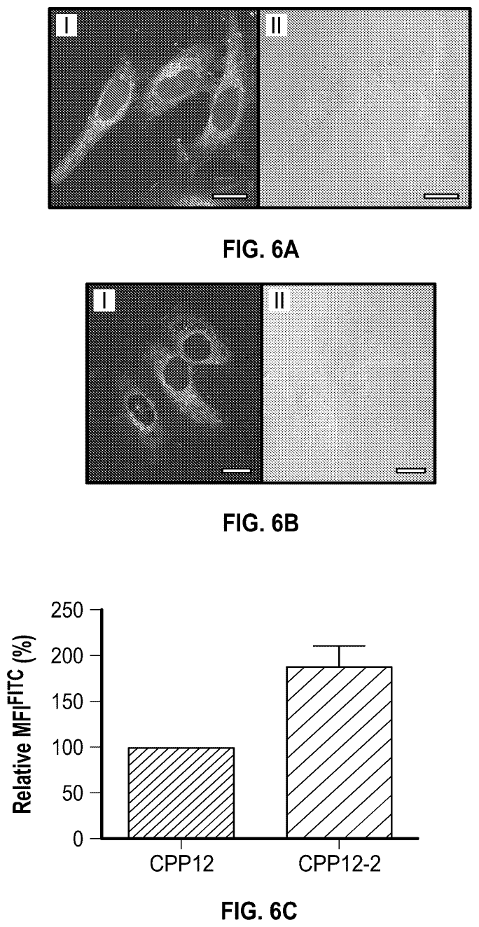

The capability of CPP12-2 to enter the cytosol of mammalian cells was confirmed by live-cell confocal microscopy of HeLa cells after treatment with 5 μM CPP12-2^{TMR} for 2 h in the presence of 1% FBS. All of the treated cells showed intense and diffuse fluorescence throughout the entire cell volume (FIG. 4A). The presence of strong TMR fluorescence inside the nucleus demonstrates that at least a fraction of the internalized CPP12-2 reached the cytosol, prior to nuclear localization. In some of the treated cells, punctate fluorescence was also visible in the cytoplasmic region and may represent the fraction of CPP12-2 that had not yet escaped from the endosomes and/or cytosolic CPP12-2 that became associated with intracellular organelles (e.g., the cytoplasmic leaflet of the endosomal membrane). CPP12^{TMR} exhibited very similar intracellular distribution (FIG. 4B). Note that under the assay condition (5 μM CPP and 1% FBS), CPP12 and CPP12-2 are expected to have similar total cellular uptake as well as endosomal escape efficiencies (Table C). HeLa cells treated with lower concentrations of CPP12^{TMR} or CPP12-2^{TMR} (0.2 and 1.0 μM) for 2 h produced predominantly punctate fluorescence in the cytoplasmic region (FIG. 5). Similar results were observed when HeLa cells were treated with fluorescein-labeled CPP12 and CPP12-2 (FIGS. 6A-C).

TABLE C
Total Cellular Uptake, Cytosolic Entry, and
Endosomal Escape efficiencies of CPP12-2^{a}

| | | | | Apparent |
|---|---|---|---|---|
| | | Total | Cytosolic | Endosomal |
| | | Cellular | Entry | Escape |
| [CPP12-2] | | Uptake | Efficiency | Efficiency |
| (μM) | [FBS] | (MFI^{TMR}, %) | (MFI^{NF}, %) | (γ, %) |
| CPP12 | 10% or 1% | 100 | 100 | 100 |
| 5.0 μM | 10% | 98 ± 11 | 375 ± 48 | 383 ± 49 |
| CPP12-2 | | | | |
| 5.0 μM | 1% | 79 ± 13 | 85 ± 7 | 108 ± 9 |
| CPP12-2 | | | | |
| 2.0 μM | 1% | 84 ± 10 | 116 ± 5 | 138 ± 6 |
| CPP12-2 | | | | |
| 1.0 μM | 1% | 88 ± 8 | 145 ± 21 | 164 ± 24 |
| CPP12-2 | | | | |
| 0.5 μM | 1% | 94 ± 10 | 186 ± 7 | 198 ± 7 |
| CPP12-2 | | | | |

^{a}All values represent the mean ± SD of at least three independent experiments and are relative to that of CPP12 (100%).

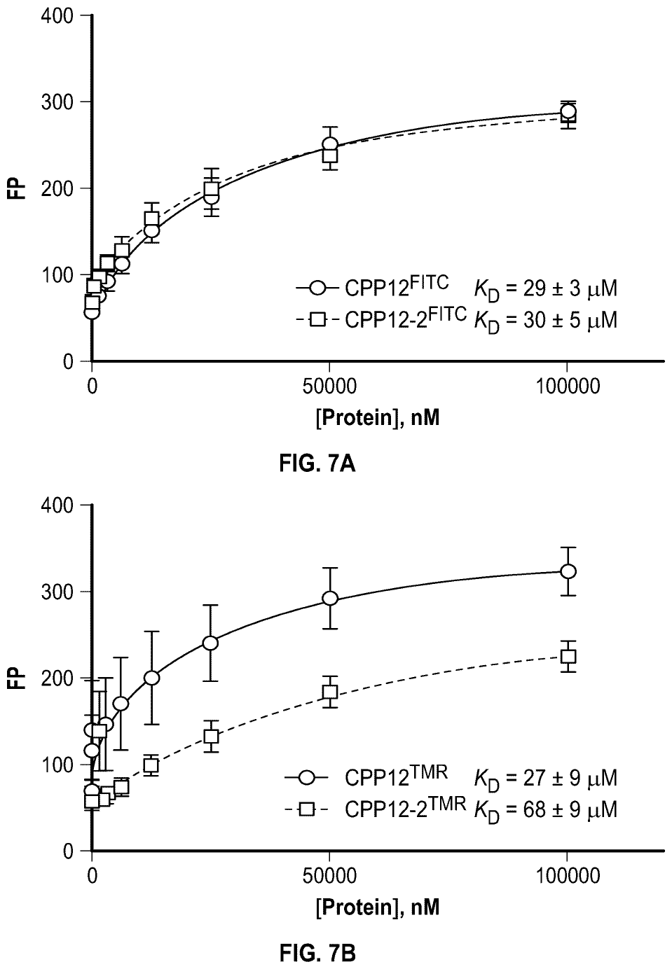

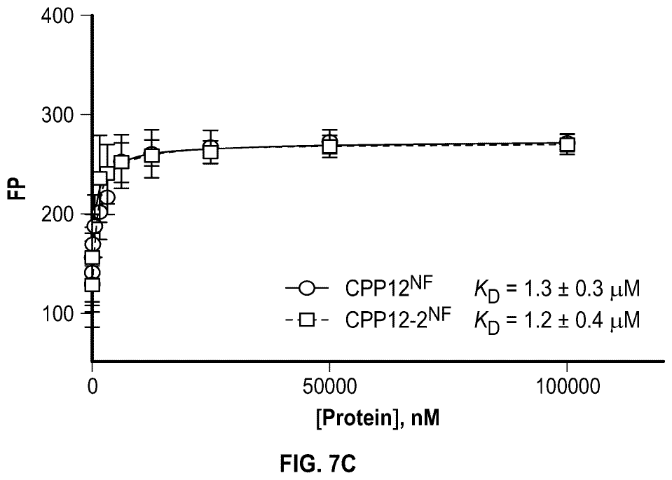

Finally, to test whether the differential sensitivity of CPP12 and CPP12-2 to serum concentration is caused by difference in binding to serum proteins, we determined the binding affinity of FITC-, NF-, or TMR-labeled CPP12 and CPP12-2 to serum proteins by fluorescence polarization (FP). Thus, CPP12^{FITC} and CPP12-2^{FITC} exhibited apparent K_{D} values of 29±3 μM and 30±5 μM, respectively, to proteins in FBS (FIGS. 7A-C). CPP12^{TMR} and CPP12-2^{TMR} showed apparent K_{D} values of 27±9 μM and 68±9 μM, while CPP12^{NF} and CPP12-2^{NF} showed apparent K_{D} values of 1.3±0.3 μM and 1.2±0.4 μM, respectively. Thus, although the dye structure can significantly affect the binding affinity of CPP12 and CPP12-2 to serum proteins, the two CPPs bind to serum proteins with very similar affinities, excluding protein binding as a significant contributor to the observed differential sensitivity of CPP12 and CPP12-2 to serum concentration.

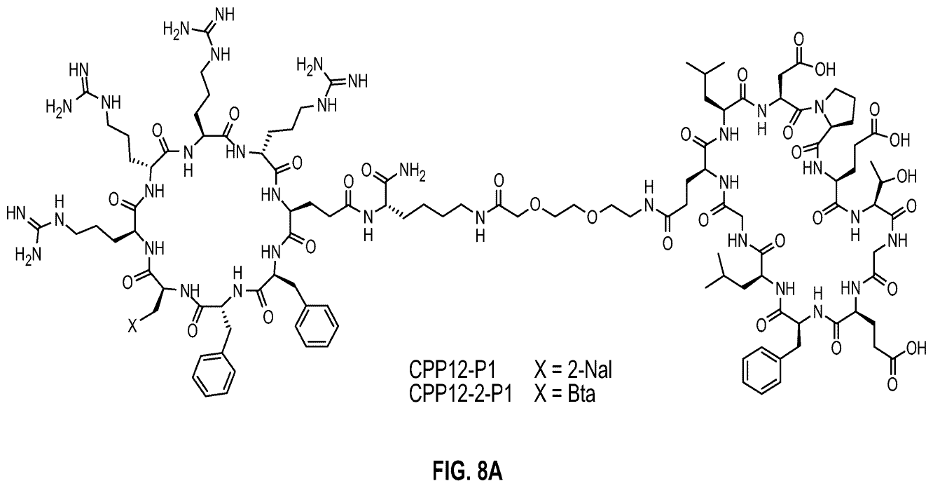

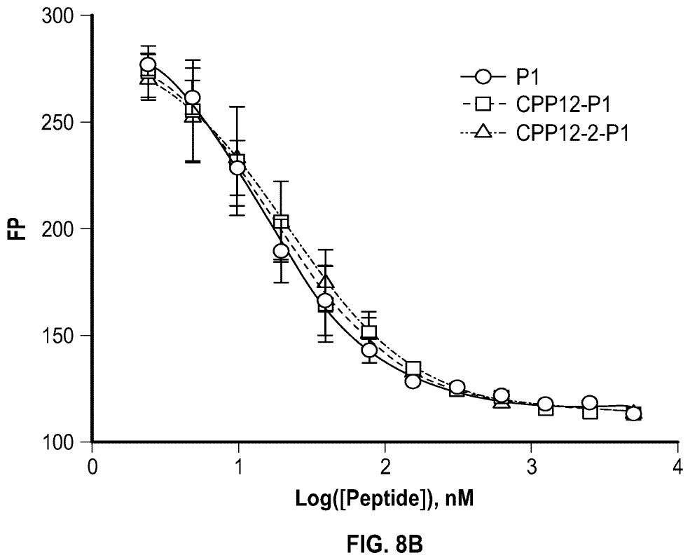

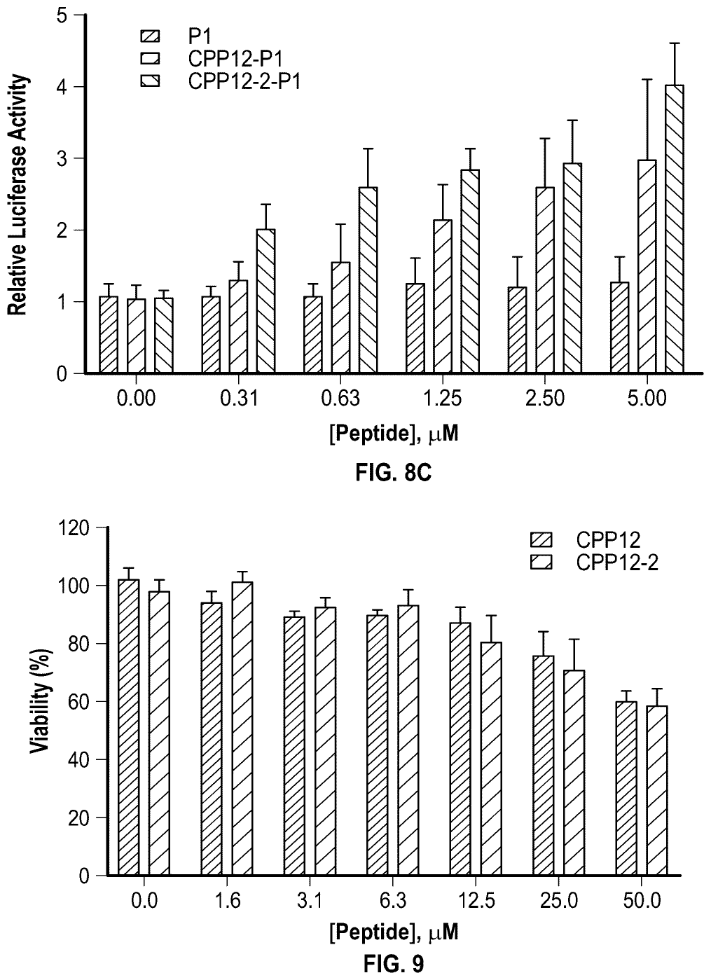

Results—Improved Cytosolic Delivery of Cyclic Peptidyl Cargo. We next tested the capacity of CPP12-2 to deliver biologically active cargo into mammalian cells. A previously reported cyclic peptidyl inhibitor against the Kelch-like ECH-associated protein 1 (Keap1)-nuclear factor erythroid-2 (Nrf2) interaction was chosen as a model peptide to test the efficacy of the new CPP. Cyclo (GQLDPETGEFL) (P1; K_{D}=18 nM)⁴³ was conjugated to CPP12 or CPP12-2 through a flexible miniPEG linker (CPP12-P1 and CPP12-2-P1; FIG. 8A) and their binding to the Kelch domain of Keap1 was tested by a fluorescence polarization (FP)-based competition assay. Peptides P1, CPP12-P1, and CPP12-2-P1 showed IC₅₀ values of 14±1, 18±1, and 22±1 nM, respectively (FIG. 8B). These data indicate that conjugation of P1 to cyclic CPPs only slightly reduced its Keap1-binding affinity. We next examined CPP12-P1 and CPP12-2-P1 for their inhibition of the intracellular Keap1-Nrf2 interaction by using an ARE reporter-HepG2 cell line, which contains a firefly luciferase gene under the transcriptional control of Nrf2.⁴⁴ Under basal conditions, Nrf2 interacts with Keap1 and is retained in the cytosol or degraded by the proteasome. However, upon blocking the Keap1-Nrf2 interaction, Nrf2 accumulates and translocates into the nucleus, inducing the expression of luciferase. As reported previously,³⁴ P1 did not result in significant increase in luciferase activity up to 5 mM concentration (FIG. 8C). On the other hand, both CPP12-P1 and CPP12-2-P1 increased the luciferase expression in a dose-dependent manner, with maximal induction of 3- and 4-fold, respectively, at the highest concentration tested (5 μM). However, CPP12-2-P1 was more efficacious than CPP12-P1 at all concentrations tested, especially at lower concentrations. For example, at 310 nM concentration CPP12-2-P1 increased the luciferase activity by ˜2-fold, whereas CPP12-P1 did not have significant effect at the same concentration. These results confirm that CPP12-2 is a more effective delivery vehicle than CPP12 at low concentrations.

Results—CPP12-2 Is Non-cytotoxic. CPP12-2 was assessed for potential cytotoxicity against HeLa cells by the 3-(4,5-dimethylthiazol-2-yl)-2,5-diphenyltetrazolium bromide (MTT) assay. Like CPP12, CPP12-2 did not reduce the viability of HeLa cells at up to 25 μM concentration (FIG. 9). However, significant loss of viability (˜40%) was observed at ≥50 μM concentration for both CPPs.

Conclusions—In this study, we have discovered a new cyclic CPP, CPP12-2, which has up to 4-fold greater cytosolic delivery efficiency than CPP12, the most potent cyclic CPP previously reported. The improved cytosolic delivery efficiency of CPP12-2 is primarily the result of more efficient endosomal escape, especially at low CPP concentrations.

A number of embodiments of the invention have been described. Nevertheless, it will be understood that various modifications may be made without departing from the spirit and scope of the invention. Accordingly, other embodiments are within the scope of the following claims.

The disclosure provides for the following example embodiments, the numbering of which is not to be construed as designating levels of importance:

Embodiment 1 relates to a cyclic peptide, comprising from 6 to 20 amino acids, wherein at least three of the amino acids are arginine; and at least three of the amino acids have a hydrophobic side chain selected from an aryl or heteroaryl, wherein the aryl and heteroaryl are optionally substituted.

Embodiment 2 relates to a cyclic peptide of Embodiment 1, comprising at least one hydrophobic amino acid having an aryl side chain and at least one hydrophobic amino acid having a heteroaryl side chain.

Embodiment 3 relates to a cyclic peptide of Embodiment 1 or 2, comprising two hydrophobic amino acid having an aryl side chain and one hydrophobic amino acid having a heteroaryl side chain.

Embodiment 4 relates to a cyclic peptide of Embodiments 1-3, comprising from 6-10 amino acids.

Embodiment 5 relates to a cyclic peptide of Embodiments 1-4, wherein the hydrophobic amino acid is selected from the group consisting of L-3-benzothienylalanine, L-4-fluorophenylalanine, D-4-fluorophenylalanine, L-1-naphthylalanine, L-2-naphthylalanine, L-2-pyridylalanine, D-2-pyridylalanine, L-4-pyridylalanine, D-4-pyridylalanine, L-phenylalanine, D-phenylalanine, L-tyrosine, D-tyrosine, and combinations thereof.

Embodiment 6 relates to a cyclic peptide of Embodiments 1-5, wherein the at least three hydrophobic amino acids are L-phenylalanine, D-phenylalanine, and L-3-benzothienylalanine.

Embodiment 7 relates to a cyclic peptide of Embodiments 1-6, wherein the at least three hydrophobic amino acids are L-phenylalanine, D-4-pyridylalanine, and L-2-napthylalanine.

Embodiment 8 relates to a cyclic peptide of Embodiments 1-7, wherein at least one hydrophobic amino acid is L-3-benzothienylalanine.

Embodiment 9 relates to a cyclic peptide of Embodiments 1-8, wherein at least one hydrophobic amino acid is D-4-pyridylalanine.

Embodiment 10 relates to a cyclic peptide of Embodiments 1-9, comprising at least one D-amino acid.

Embodiment 11 relates to a cyclic peptide of Embodiments 1-10, comprising at least two D-amino acids.

Embodiment 12 relates to a cyclic peptide of Embodiments 1-11, comprising at least three D-amino acids.

Embodiment 13 relates to a cyclic peptide of Embodiments 1-12, comprising at least two consecutive amino acids having the same chirality.

Embodiment 14 relates to a cyclic peptide of Embodiments 1-13, wherein the two consecutive amino acids having the same chirality are arginine and an amino acid having a hydrophobic side chain.

Embodiment 15 relates to a cyclic peptide of Embodiments 1-14, comprising at least two consecutive amino acids having alternating chirality.

Embodiment 16 relates to a cyclic peptide of Embodiments 1-15, comprising at least three consecutive amino acids having alternating chirality.

Embodiment 17 relates to a cyclic peptide of Embodiments 1-16, comprising at least four consecutive amino acids having alternating chirality.

Embodiment 18 relates to a cyclic peptide of Embodiments 1-17, comprising at least five consecutive amino acids having alternating chirality.

Embodiment 19 relates to a cyclic peptide of Embodiment 15, wherein the at least two consecutive amino acids having alternating chirality are hydrophobic amino acids.

Embodiment 20 relates to a cyclic peptide of Embodiment 16, wherein the at least three consecutive amino acids having alternating chirality are hydrophobic amino acids.

Embodiment 21 relates to a cyclic peptide of Embodiments 1-20, comprising at least three arginines.

Embodiment 22 relates to a cyclic peptide of Embodiments 1-21, comprising at least four arginines.

Embodiment 23 relates to a cyclic peptide of Embodiments 1-22, wherein the at least two arginines are consecutive.

Embodiment 24 relates to a cyclic peptide of Embodiment 21, wherein the at least three arginines are consecutive.

Embodiment 25 relates to a cyclic peptide of Embodiment 22, wherein the at least four arginines are consecutive.

Embodiment 26 relates to a cyclic peptide of Embodiment 24, wherein at least two of the consecutive arginines have alternating chirality.

Embodiment 27 relates to a cyclic peptide of Embodiment 24, wherein at least three of the consecutive arginines have alternating chirality.

Embodiment 28 relates to a cyclic peptide of Embodiment 25, wherein at least four of the consecutive arginines have alternating chirality.

Embodiment 29 relates to a cyclic peptide of Embodiments 1-28, comprising a glutamine.

Embodiment 30 relates to a cyclic peptide of Embodiments 1-29, comprising at least one amino acid selected from the group consisting of cysteine, glutamine, 2,3-diaminopropionic acid, ornithine, lysine, serine, aspartic acid, glutamic acid, asparagine, and tryptophan.

Embodiment 31 relates to a cyclic peptide of Embodiment 1, having a structure of

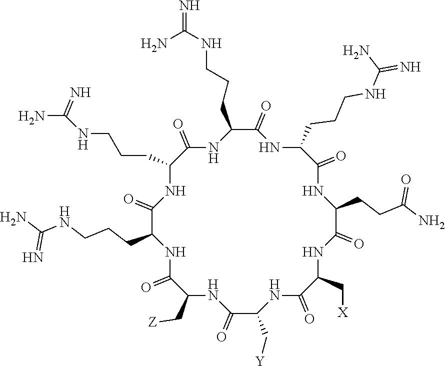

wherein each of X, Y, and Z independently comprise aryl or heteroaryl, wherein the aryl and heteroaryl are optionally substituted.

Embodiment 32 relates to a cyclic peptide of Embodiment 1, having a structure of:

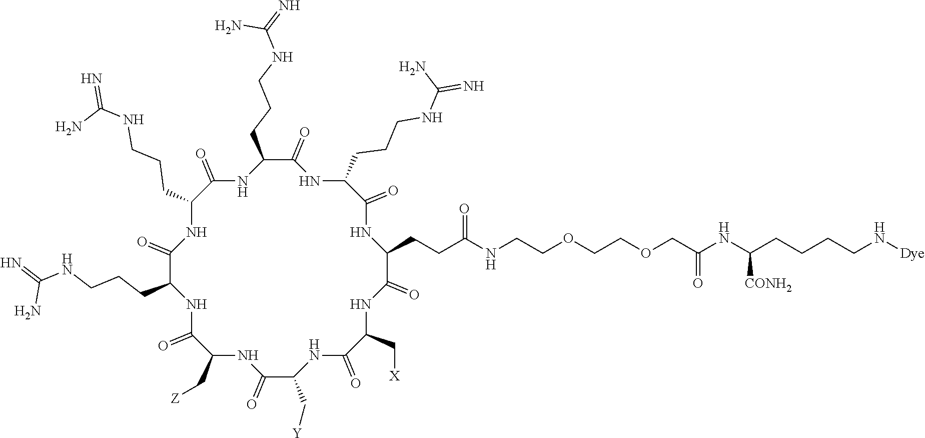

wherein each of X, Y, and Z independently comprise aryl or heteroaryl, wherein the aryl and heteroaryl are optionally substituted.

Embodiment 33 relates to a cyclic peptide of Embodiment 31 or 32, wherein X, Y, and Z are independently selected from C₆₋₁₄ aryl or C₅-C₁₅ heteroaryl having from 1 to 5 heteroatoms selected from N, O, or S.

Embodiment 34 relates to a cyclic peptide of Embodiment 31 or 32, wherein two of X, Y, and Z are independently C₆₋₁₄ aryl and one of X, Y, and Z are C₅-C₁₅ heteroaryl having from 1 to 5 heteroatoms selected from N, O, or S.

Embodiment 35 relates to a cyclic peptide of Embodiment 31 or 32, wherein X, Y, and Z are independently phenyl, naphthyl, pyridyl, or benzothienyl.

Embodiment 36 relates to a cyclic peptide of Embodiment 31 or 32, wherein X, Y, and Z are independently selected from the group consisting of phenyl, pyridyl, and naphthyl.

Embodiment 37 relates to a cyclic peptide of Embodiment 31 or 32, wherein X is phenyl, Y is phenyl, and Z is benzothienyl.

Embodiment 38 relates to a cyclic peptide of Embodiment 31 or 32, wherein X is phenyl, Y is pyridyl, and Z is naphthyl.

Embodiment 39 relates to a cyclic peptide of Embodiment 31 or 32, wherein X, Y, and Z comprise a side chain from an amino acid independently selected from the group consisting of 3-benzothienylalanine, 4-fluorophenylalanine, 1-naphthylalanine, 2-naphthylalanine, 2-pyridylalanine, 4-pyridylalanine, phenylalanine, tyrosine, and combinations thereof.

Embodiment 40 relates to a cyclic peptide of Embodiments 1-39, wherein the cyclic peptide has a relative cytosolic delivery efficiency which is improved by about 110% to about 400% compared to cyclo(FfΦRrRrQ).

Embodiment 41 relates to a cyclic peptide of Embodiments 1-40, wherein the cyclic peptide has a relative cytosolic delivery efficiency which is improved by about 375% compared to cyclo(FfΦRrRrQ).

Embodiment 42 relates to a cyclic peptide of Embodiments 1-41, wherein the cyclic peptide has a relative cytosolic delivery efficiency which is improved by about 115% compared to cyclo(FfΦRrRrQ).

Embodiment 43 relates to a cyclic peptide of Embodiments 1-42, wherein the cyclic peptide has an endosomal escape efficiency, γ, which is improved by about 108% to about 400% compared to cyclo(FfΦRrRrQ).

Embodiment 44 relates to a cyclic peptide of Embodiments 1-43, wherein the cyclic peptide has an endosomal escape efficiency, γ, which is improved by about 108% compared to cyclo(FfΦRrRrQ).

Embodiment 45 relates to a cyclic peptide of Embodiments 1-44, wherein the cyclic peptide has an endosomal escape efficiency, γ, which is improved by about 383% compared to cyclo(FfΦRrRrQ).

Embodiment 46 relates to a cyclic peptide of Embodiment 1-45, having an amino acid sequence selected from the group consisting of:

(i)
Phe-phe-(1-Nal)-Arg-arg-Arg-arg-Gln
(ii)
Phe-phe-(Bta)-Arg-arg-Arg-arg-Gln
(iii)
Phe-(D-Fpa)-(2-Nal)-Arg-arg-Arg-arg-Gln
(iv)
Phe-(D-2-Pya)-(2-Nal)-Arg-arg-Arg-arg-Gln
(v)
Phe-(D-4-Pya)-(2-Nal)-Arg-arg-Arg-arg-Gln
(vi)
Phe-(D-Tyr)-(2-Nal)-Arg-arg-Arg-arg-Gln
(vii)
Fpa-(D-Phe)-(2-Nal)-Arg-arg-Arg-arg-Gln
(viii)
(2-Pya)-phe-(2-Nal)-Arg-arg-Arg-arg-Gln
(ix)
(4-Pya)-phe-(2-Nal)-Arg-arg-Arg-arg-Gln
(x)
Tyr-phe-(2-Nal)-Arg-arg-Arg-arg-Gln

Embodiment 47 relates to a cyclic peptide of Embodiments 1-46 having an amino acid sequence of Phe-phe-(Bta)-Arg-arg-Arg-arg-Gln.

Embodiment 48 relates to a cyclic peptide of Embodiments 1-47 having an amino acid sequence of Phe-(D-4-Pya)-(2-Nal)-Arg-arg-Arg-arg-Gln.

Embodiment 49 relates to a cyclic peptide of Embodiments 1-48 comprising a cargo.

Embodiment 50 relates to a cyclic peptide of Embodiment 49, wherein the cargo moiety is selected from the group consisting of a detectable moiety, a targeting moiety, a therapeutic moiety, or any combination thereof.

Embodiment 51 relates to a cyclic peptide of Embodiment 49, wherein the cargo is coupled to a side chain of an amino acid of the cyclic peptide.

Embodiment 52 relates to a cyclic peptide of Embodiment 49, wherein the cargo is coupled to a glutamine side chain of the CPP.

Embodiment 53 relates to a method of treating a disease or pathology in a subject in need comprising administering to the subject a cyclic peptide of any one of Embodiments 1-52.

Embodiment 54 relates to a method of treating cancer in a subject in need comprising administering to the subject a cyclic peptide of any one of Embodiments 1-52.

We claim:

1. A cyclic peptide of structure 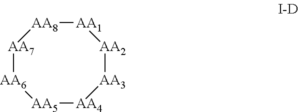
or a pharmaceutically acceptable salt thereof;
wherein
each of AA₁, AA₂, AA₃, AA₄, AA₅, AA₆, AA₇, and AA₈, are independently selected from an amino acid, and there is a cargo moiety coupled to a side chain of an amino acid selected from AA₁, AA₂, AA₃, AA₄, AA₅, AA₆, AA₇, and AA₈;
two of AA₁, AA₂, AA₃, AA₄, AA₅, AA₆, AA₇, and AA₈ are arginine, with the remaining amino acids other than arginine;
five of AA₁, AA₂, AA₃, AA₄, AA₅, AA₆, AA₇, and AA₈ are independently selected consecutive hydrophobic amino acids; and
the cargo moiety comprises a linker moiety, a therapeutic moiety, and a targeting moiety.

2. The cyclic peptide of claim 1, wherein at least one of AA₁, AA₂, AA₃, AA₄, AA₅, AA₆, AA₇, and AA₈ is phenylalanine.

3. The cyclic peptide of claim 1, wherein two of AA₁, AA₂, AA₃, AA₄, AA₅, AA₆, AA₇, and AA₈ are phenylalanine.

4. The cyclic peptide of claim 1, wherein at least one of AA₁, AA₂, AA₃, AA₄, AA₅, AA₆, AA₇, and AA₈ is glycine.

5. The cyclic peptide of claim 1, wherein at least two of AA₁, AA₂, AA₃, AA₄, AA₅, AA₆, AA₇, and AA₈ are glycine.

6. The cyclic peptide of claim 1, wherein three of AA₁, AA₂, AA₃, AA₄, AA₅, AA₆, AA₇, and AA₈ are glycine.

7. The cyclic peptide of claim 1, wherein an arginine is adjacent to a hydrophobic amino acid.

8. The cyclic peptide of claim 1, wherein both arginines are adjacent to hydrophobic amino acids.

9. The cyclic peptide of claim 7, wherein the adjacent hydrophobic amino acid is glycine

10. The cyclic peptide of claim 8, wherein the adjacent hydrophobic amino acids are glycine.

11. The cyclic peptide of claim 10, wherein the therapeutic moiety is an antisense compound.

12. The cyclic peptide of claim 1, wherein the therapeutic moiety is an antisense compound.


SEQUENCE LISTING (8 sequences)

SEQ ID NO: 1 - protein, 5 aa, synthetic construct
RRRRR

SEQ ID NO: 2 - protein, 5 aa, synthetic construct
AAAAA

SEQ ID NO: 3 - protein, 4 aa, synthetic construct
FFFF

SEQ ID NO: 4 - protein, 7 aa, synthetic construct
AAAAAAA

SEQ ID NO: 5 - protein, 5 aa, synthetic construct
RARAR

SEQ ID NO: 6 - protein, 5 aa, synthetic construct
DADAD

SEQ ID NO: 7-8 - skipped (2 entries)